(19) United States
(12) Patent Application Publication
(10) Pub. No.: US 2018/0271397 A1
(43) Pub. Date: Sep. 27, 2018

(54) MODEL OF ELECTRIC CONDUCTIVITY OF WHITE MATTER

(71) Applicant: Government of the United States as Represented by the Secretary of the Air Force, Wright-Patterson AFB, OH (US)

(72) Inventors: Noel D. Montgomery, Adkins, TX (US); Jack L. Lancaster, San Antonio, TX (US)

(73) Assignee: Government of the United States as Represented by the Secretary of the Air Force, Wright-Patterson AFB, OH (US)

(21) Appl. No.: 15/933,842

(22) Filed: Mar. 23, 2018

Related U.S. Application Data
(60) Provisional application No. 62/476,072, filed on Mar. 24, 2017.

(51) Int. Cl. A61B 5/055; A61B 5/00; G06T 7/00; G01R 33/50

(52) U.S. Cl. CPC A61B 5/055; A61B 5/4064; G01R 33/56341; G06T 7/0012; G01R 33/50; A61B 5/0042

(57) ABSTRACT

A model and method for mapping tissue conductivity. The method of mapping tissue conductivity includes estimating an extra-axonal fraction using T1- and T2-weighted magnetic resonance images. Longitudinal and transverse flow directions are assigned using diffusion-weighted magnetic resonance images.

Pursuant to 37 C.F.R. § 1.78(a)(4), this application claims the benefit of and priority to prior filed co-pending Provisional Application Ser. No. 62/476,072, filed Mar. 24, 2017, which is expressly incorporated herein by reference, in its entirety.

### RIGHTS OF THE GOVERNMENT

The invention described herein may be manufactured and used by or for the Government of the United States for all governmental purposes without the payment of any royalty.

### FIELD OF THE INVENTION

The present invention relates generally to brain conductivity and, more particularly, to methods of mapping brain conductivity.

### BACKGROUND OF THE INVENTION

Electric fields exert a force on charged particles of a material. The resulting force may polarize bound charges to create induced dipoles, cause rotation of permanent dipoles, drift or flow of unbound charges, or combinations thereof. Materials where charge transport is the dominant process are called conductors; materials that are susceptible to the formation of induced dipoles and rotation of permanent dipoles (like biological tissue) are called dielectrics.

Magnetic fields in materials affect magnetic dipoles in a manner that depends on material type. Biological tissues lack a significant amount of magnetic dipoles at the macroscopic level, so the macroscopic magnetic response within biological tissues is nearly identical to the magnetic response free space. At a microscopic level, a paramagnetic response of nuclear magnetic moments from the proton of hydrogen in water molecules forms the basis for clinical Magnetic Resonance Imaging (“MRI”). Under ambient conditions, the dipoles of the protons of the water molecules within biological tissue are randomly oriented and have no net magnetization. Indeed, even when placed within a strong main magnetic field, the net magnetization within biological tissue is miniscule.

The response of materials to electric fields may be quantified by the complex conductivity tensor:

σ*=σᵣ−iσᵢ Equation 1,

where σᵣ is the real portion of a conductivity tensor, σᵢ is the imaginary part (where σᵢ=ω∈₀∈′), ω is the angular frequency, ∈₀ is the permittivity of free space, and ∈′ is the relative permittivity tensor. The complex conductivity tensor formalism is driven by the dispersive response to a stimulus: the response of bound charges (polarization) to the response of free charges (displacement, such as by drift or flow) varying with frequency. The relative permittivity, σᵣ, (also called the dielectric constant) quantifies the polarizability of the dielectric material. In other words, the relative permittivity is the ease with which an electric field may form induced dipoles and rotate permanent dipoles. Thus, the magnitude of σᵣ is a measure of the ease with which currents may flow in the material. Within biological tissue, current is caused primarily by a flow of ions.

Ohm's law may be applied to determine current flow in a material when an electric field is applied at a particular frequency, ω:

Jₜₒₜ=J_{c}+J_{d}=σᵣE−iσᵢE Equation 2.

The sources of current that contribute to a total current density, Jₜₒₜ, are J_{c}, the conductive current density (arising from the movement of charges) and J_{d}, the displacement current density (arising from polarization effects).

The relative importance of ionic conductivity and polarizability is dependent on the properties of the medium and the frequency of the fields in the medium. The relative permittivity and conductivity within biological tissue, for example, white matter of the brain, varies with frequency. For instance, in white matter at 10 kHz, σᵢ/σᵣ≈10, and at 1 kHz, σᵢ/σᵣ≈16. Therefore, for frequencies less than about 10 kHz the magnitude of the polarization current comprises 10% or less of the total current. Such tissue is well characterized by a measurement of conductivity below approximately 10 kHz for applications. For frequencies above 10 kHz, the conductive current density becomes predominant; for frequencies in the microwave range, conductive current density is often neglected.

Such current conductivity and relative permittivity evaluation is useful in understanding, treating, and managing certain medical conditions. An estimated 2.2 million people in the United States have epilepsy, and as many as 30% of those epileptic patients are refractory to pharmaceutical treatments that may become candidates for surgical resection of the epileptogenic focus. Over 8000 such surgeries were reported to have been performed worldwide from 1985 to 1990, and the rate is believed to be growing.

Medical practitioners and scientists rely on voxelized models of human tissue electromagnetic properties to calculate a location of electrical activity, to determine energy deposition, and to determine current flows in the human body. Voxelized models comprise a large number of small voxels (volumized pixels) that represent tissue properties contained therein. In these models, each voxel is assigned a set of electromagnetic properties based on the tissue type represented thereby. A differential equation may be solved for each voxel based on the voxel characteristics and the characteristics of surrounding voxels. Such voxelized models of tissue permittivity and conductivity may be used to determine location of current sources or, more specifically for example, to select targets for treatment or predict adverse effect limits on bulk currents.

Most conventional voxelized models assume all tissues have isotropic (conductivity having no net direction). However, certain tissues, such as white matter and skeletal muscle, are clearly anisotropic (conductivity having a net direction). Techniques for estimating anisotropic properties that rely on water diffusion as a surrogate for electric conductivity have been developed but are not successfully validated.

Tissue conductivity, for the purposes of electrophysiology, was initially measured using transcranial electric stimulation (“TES”), transcranial magnetic stimulation (“TMS”), and electromagnetic and electrical safety dosimetry. Few papers exist documenting anisotropic conductivity measurements as most research presumed conductivity to be isotropic. These conventional techniques either measured bulk, isotropic conductivity or reported results without indicating orientation of the tissue structure with respect to the measurement direction. For anisotropic tissues, conductivity should be measured in multiple directions, such as a four-electrode measurement (“4EM”) system, with both magnitude and direction reported.

More recently, electrophysiologists use diagnostic modalities such as electroencephalography (“EEG”), electrocardiography (“ECG”), and electromyography (“EMG”) to measure electric potentials or currents at a surface of the body to assess underlying electrophysiology. EEG is used clinically, such as in the diagnosis of epilepsy, and in research to link temporal aspects of brain activity to cognition and behavior. The EEG modality suffers from an inability to localize a source of brain activity, partly because accurate conductivity maps are not available.

TMS uses a large pulsed magnetic field to induce currents in the brain at specific targets that then cause groups of neurons to fire and a physiological change. Current flows are determined from the electric field induced by the pulsed magnetic field and the underlying conductivity pattern of the brain tissue. Conventional dosimetric planning methods for TMS include boundary element method calculations, which provide fairly good localization of currents. However, individualized conductivity maps would improve TMS dosimetry and help to reduce variability in the outcome of the experiments. For instance, when anisotropy is considered, TMS model results have varied as much as 10% to 40%.

Electromagnetic safety researchers rely on voxelized models to predict current flow and energy deposition from an exposure to external fields and contact currents. Most of the conventional models in use today for low frequency field and current exposures are derived from a single individual by the visible human project. Much work has been done to generalize that tissue information to the population, including the creation of female and child models, however, all of these models have assumed isotropic conductivity in all tissue.

Several groups have attempted to map anisotropy of tissue conductivity using diffusion tensor imaging (“DTI”), which correlates direction and magnitude of water diffusion with image signal. Most commonly, linear scaling is applied to three diffusion tensor eigenvalues (dᵥ) to approximate three conductivity tensor eigenvalues (σᵥ):

σᵥ=Adᵥ+B Equation 3,

where A and B, the slope, and the intercept of the linear model were calibrated using historical measurements of tissue conductivity.

Several approaches have been taken to evaluate the conductivity tensor eigenvalues. One is a statistical micromechanics approach to develop a cross product relationship between the diffusion tensor and the conductivity tensor (σᵥ=0.844dᵥ±0.0545). Another approach is the assigning a magnitude of conductivity along each eigenvector such that the average is equivalent to the isotropic conductivity from previous measurements in literature (σᵥ=0.21dᵥ). Still another approach is to artificially constrain the anisotropy of the predicted conductivity to match previously calculated anisotropy values measured using a uniform E-field to indicate whether diffusion eigenvalues may not be a good predictor of anisotropic conductivity. Yet another approach includes basing conductivity of tissue on a fraction of extracellular diffusion derived from a bi-exponential model of diffusion at higher b values. Another approach is assigning three compartments of signal to cells oriented in three orthogonal directions with a fourth to glial cells (assumed to be spherical and, therefore, isotropic).

Another promising approach is to map conductivity and permittivity in brain tissue. Electric properties tomography (“EPT”), often called B1 imaging, uses perturbations in the magnetic field of an RF pulse to estimate conductivity and permittivity of brain tissue. A limitation of B1 imaging is that conductivity and permittivity maps are limited by the Larmor frequency of the MRI (42.6 MHz/Tesla, thus 127.71 MHz for proton imaging at 3.0 Tesla).

Magnetic Resonance Electrical Impedance Tomography (“MREIT”) images present a pattern of current density or magnetic flux density resulting from external current injected through surface electrodes. MREIT is a promising approach to imaging conductivity and has been used to image conductivity in both animals and humans. Conventionally, MREIT has been used to map isotropic conductivity because earlier methods involved rotating the patient to three orientations. While anisotropic conductivity mapping using a single orientation is theoretically possible, the process would require at least seven currents for complete reconstruction. Isotropic conductivity maps from MREIT combined with DTI have been used to estimate anisotropic conductivity in tissue.

One significant value to MRI-based conductivity mapping is that it allows researchers to use “subject-specific” models of conductivity to predict energy deposition and current flows for dosimetry. Research findings based on subject-specific models will provide more insight concerning the effect of exposures on the human population.

As a result, it would be of great benefit for MRI-based methods to be further developed in a manner that yield subject specific models of tissue conductivity for both research and medical treatment applications.

### SUMMARY OF THE INVENTION

The present invention overcomes the foregoing problems and other shortcomings, drawbacks, and challenges of the conventional brain conductivity maps. While the invention will be described in connection with certain embodiments, it will be understood that the invention is not limited to these embodiments. To the contrary, this invention includes all alternatives, modifications, and equivalents as may be included within the spirit and scope of the present invention.

According to one embodiment of the present invention a method of mapping tissue conductivity includes estimating an extra-axonal fraction using T1- and T2-weighted magnetic resonance images. Longitudinal and transverse flow directions are assigned using diffusion-weighted magnetic resonance images.

Other embodiments of the present invention are directed to methods of mapping biological conductivity and include acquiring a plurality of T1-weighted images of a biological tissue, a plurality of T2-weighted images of the biological tissue, and a plurality of diffusion-weighted images of the biological tissues. An extra-axonal fraction of a region of interest using the pluralities of T1- and T2-weighted images. A longitudinal direction of anisotropy and a transverse direction of anisotropy for the region of interest are estimated using the plurality of diffusion-weighted images.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a schematic illustration of a human brain cut along midline in the sagittal plane.

FIG. 2 is a schematic illustration of a neuron.

FIGS. 3A-3C are perspective views of three models of axonal arrangement.

FIG. 4 is a flowchart illustrating a method of measuring conductivity using magnetic resonance imaging according to an embodiment of the present invention.

FIG. 5 illustrates an exemplary spin echo sequence.

FIG. 6 illustrates an exemplary inversion recovery spin echo sequence.

FIG. 7 illustrates an exemplary gradient echo sequence.

FIG. 8 illustrates an exemplary diffusion-weighted sequence.

FIG. 9 graphically illustrates exemplary eigenvalues and eigenvectors derived from diffusion tensor images according to an embodiment of the present invention.

FIG. 9A illustrates the axes of FIG. 9 with anatomical markers.

FIG. 10 is a schematic illustration of a computer suitable for use with embodiments of the present invention.

FIG. 11 graphically illustrates mass of brain cholesterol of pigs as a function of age.

FIG. 12 graphically illustrates calibration curves for the three pool model when imaging pig brain at 3 T.

FIGS. 13-15 graphically illustrate predicted potentials versus voxel size for isotropic infinite space (FIG. 13) and for longitudinal, and transverse probe orientations in an infinite anisotropic space (FIGS. 14 and 15).

FIGS. 16-18 graphically compare model predictions at increasing depths.

FIG. 19 graphically illustrates the predicted response of the 4EM system as the probe moves from 3 mm to 7 mm in depth.

FIG. 20 graphically illustrates the impact of probe position error on 4EM response.

FIG. 21 is an electrical schematic representing an electrode circuit for measuring current.

FIG. 22 graphically illustrates the calibration of the 4EM system with electrodes at increasing depths.

FIG. 23 graphically illustrates the model of slope of calibration curve versus depth.

FIGS. 24 and 25 graphically illustrate conductivity measurement data, non depth-corrected and depth-corrected, respectively.

FIG. 26 is an exemplary T2-weighted image of a sagittal slice of a pig brain at the corpus callosum.

FIG. 27 graphically illustrates the impact of heterogeneity at the interface between two regions of conductive media with differing uniform conductivities.

FIG. 28 graphically illustrates the effect of a centrally-located air boundary.

FIGS. 29-32 graphically compare estimated and measured conductivities.

FIGS. 33-35 graphically illustrate the correlation of conductivity measured by 4EM and by methods according to embodiments of the present invention.

FIGS. 36-39 graphically illustrate Bland-Altman analysis.

FIGS. 40 and 41 graphically compare transverse and longitudinal conductivities from three modeling methodologies.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

Additional information, features, and descriptions may be found in N. D. MONTGOMERY, “Model of Electric Conductivity of White Matter Using Magnetic Resonance Imaging Data,” A Dissertation Presented to the Faculty of The University of Texas Health Science Center at San Antonio, Graduate School of Biomedical Sciences, and The University of Texas at San Antonio (2016) (hereafter referred to as “the Dissertation”). The disclosure of the Dissertation is incorporated herein by reference, in its entirety.

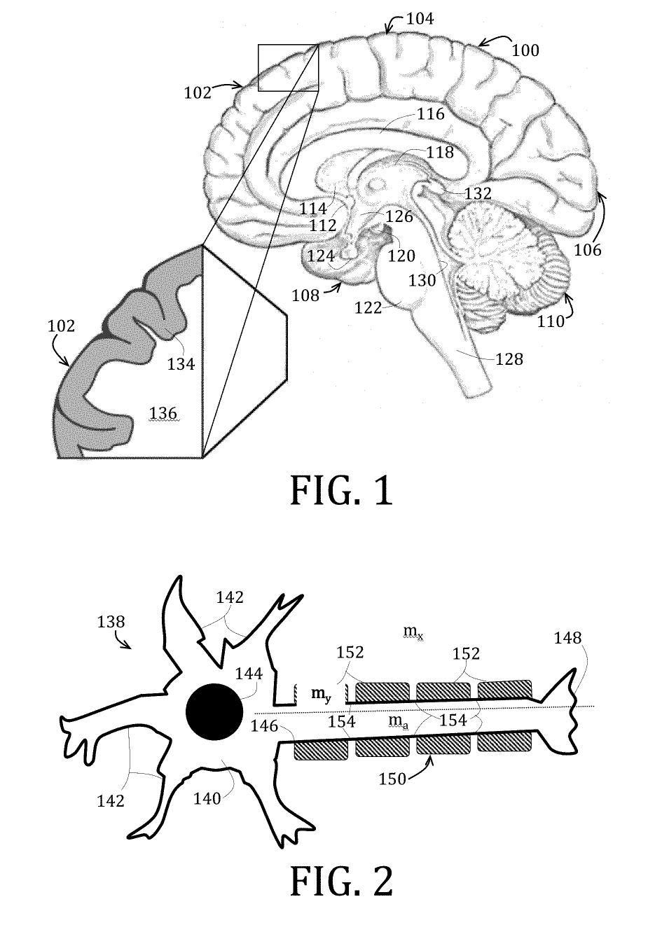

Referring now to the figures, and in particular to FIG. 1, a human brain 100 is shown in cross-section along a midline, sagittal plane. While embodiments described herein will reference brain tissue, it would be understood that certain embodiments may be applied to other tissues and that such reference made herein to brain tissue is for illustration only. Illustrated anatomy of FIG. 1 includes the frontal lobe 102, the parietal lobe 104, the occipital lobe 106, the temporal lobe 108, and the cerebellum 110, generally. More specifically, FIG. 1 also illustrates the anterior commissure 112, the septum pellucidum 114, the corpus callosum 116, the thalamus 118, the mammillary body 120, the pons 122, the pituitary 124, the hypothalamus 126, the medulla oblongata 128, the fourth ventricle 130, and the pineal body 132.

A portion of the front lobe 102 of FIG. 1 is enlarged to illustrate grey matter 134 and white matter 136. Differentiation of grey and white matter 134, 136 is based on neuronal components comprising each. And so with reference now to FIG. 2, a neuron 138 is shown and includes a cell body 140 having a plurality of dendrites 142 extending therefrom and a nucleus 144 therein. At least one axon 146 (only one axon illustrated) extends from the cell body 140 and terminates with terminal branches 148 forming synapses (not shown) with a dendrite of a subsequent neuron (not shown). The axon 146 includes a myelin sheath 150 comprising Schwann cells (not shown). Segments 152 of the myelin sheath 150 are separated by Nodes of Ranvier 154. Grey matter 134 of the brain 100 is primarily comprised of the cell bodies 140 of neurons 138 while white matter 136 of the brain 100 is primarily comprised of the myelinated axons 146.

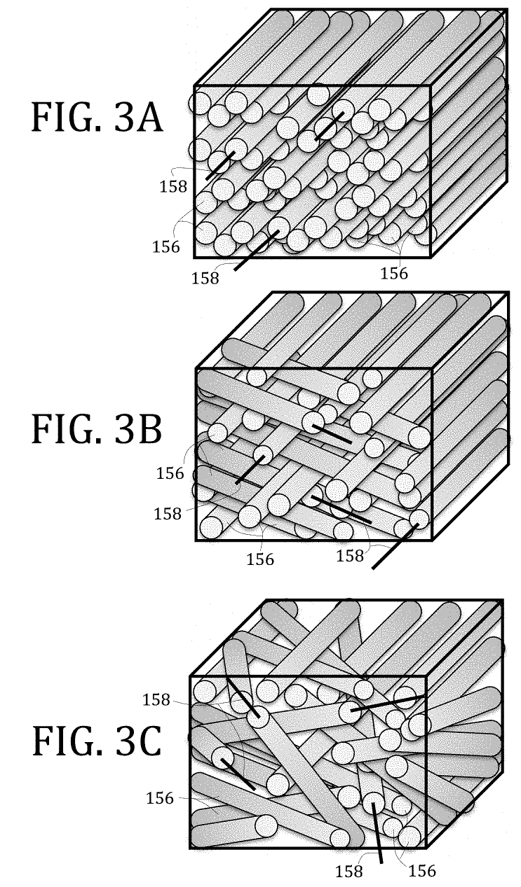

Bulk fibrous tissues, such as white matter 136, may be modeled by one of three arrangements illustrated in FIGS. 3A-3C. The model of FIG. 3A illustrates arrangements whereby each fiber 156 has a lengthwise central axis 158 and each lengthwise central axis 158 is oriented in substantially the same direction. FIG. 3B illustrates a model where the axis lengthwise central axis 158 of each fiber 156 is randomly oriented within a single plane. FIG. 3C illustrates a model where the lengthwise central axis 158 of each fiber 156 is randomly oriented in all three dimensions. White matter 136 (FIG. 1) of the central corpus callosum 116 (FIG. 1) is characterized by bundled myelinated axons in extracellular fluid arranged in a manner similar to the model of FIG. 3A. Therefore, the geometry of the axons 146 (FIG. 2) within the corpus callosum 116 (FIG. 1) is amenable to estimation using the model of FIG. 3A. Other models may be used to represent other tissues in a manner that is understood by those of ordinary skill in the art having the benefit of the disclosure made herein.

Bulk ionic conductivity of white matter 136 (FIG. 1) is thought to occur extracellularly as myelin 150 (FIG. 2) and the axonal membrane (not shown) are insulators. Conductivity within white matter 136 (FIG. 1) due to ion channel currents is thought to be a very small fraction of current in the bulk medium. As such, and using the anisotropic model of FIG. 3A, the anisotropic conductivity tensor can be described as including an estimate of extracellular volume, information about conductivity of extracellular fluid, and a measure of directionality. For example, ion conductivity that is parallel to a long axis (dashed lined of FIG. 2) of the axon 146 (FIG. 2) may be expressed as:

σ_{L}=σa Equation 4,

where a is extracellular fractional volume, and σ is conductivity of the extra-axonal volume in the absence of axons. Conductivity in a direction that is transverse to an axon may be expressed as:

$${σ}_{T} = \frac{σ a}{{λ}^{3}} , \qquad Equation 5$$

where λ is the tortuosity. Tortuosity is a ratio of the mean path length of a particle through an array of cylinders (here, the axons) (L̅_̅{̅A̅B̅}̅) to a path length of the particle not constrained by the array of cylinders (i.e., again, the axons) in the medium (L_{c}):

$$λ = \frac{\overline{{L}_{AB}}}{{L}_{C}} . \qquad Equation 6$$

Based on the foregoing, a model for tortuosity for a group of randomly overlapping, parallel cylinders (such as illustrated in FIG. 3A) thus becomes:

$$λ ( = {η}^{b} ) = {( \frac{1 - {ϵ}_{p}}{a - {ϵ}_{p}} )}^{c} , \qquad Equation 7$$

where ∈ₚ is a percolation threshold (a fraction of extracellular volume where flow is no longer possible), and c is a fitting parameter. For a bundle of aligned cylinders, ∈ₚ=0.33 and c=0.707 for diffusion transverse to the fiber direction has conventionally been used.

To model white matter conductivity tensor, Equation 7 may be used along with Equations 4 and 5 to predict longitudinal and transverse conductivity. The conductivity of extracellular fluid (∈_{elec}) in neural tissue may be presumed to be the similar to the conductivity of cerebrospinal fluid (“CSF”).

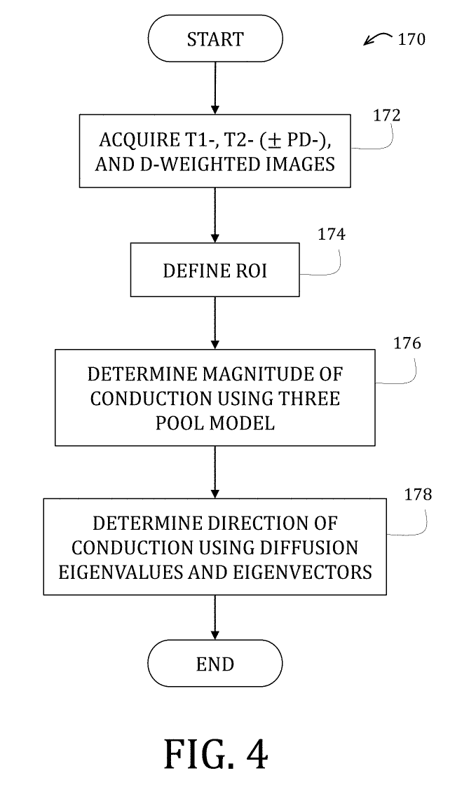

Referring now to FIG. 4, a method 170 of using MRI for estimating extra-axonal fraction and assigning longitudinal and transverse directions is described. Methods according to embodiments of the present invention may be used to overcome difficulties associated with conventional brain conductivity maps by incorporating anisotropies. MRI is a radiological imaging modality that utilizes magnetic field effects of protons (more particularly, the protons of water) of the imaged anatomy. Three main contrast mechanisms are utilized in MRI: T1 (spin-lattice relaxation), T2 (spin-spin relaxation), and PD (proton density). Both T1 and T2 are a function of the proton's environment; PD is a function of a number of protons.

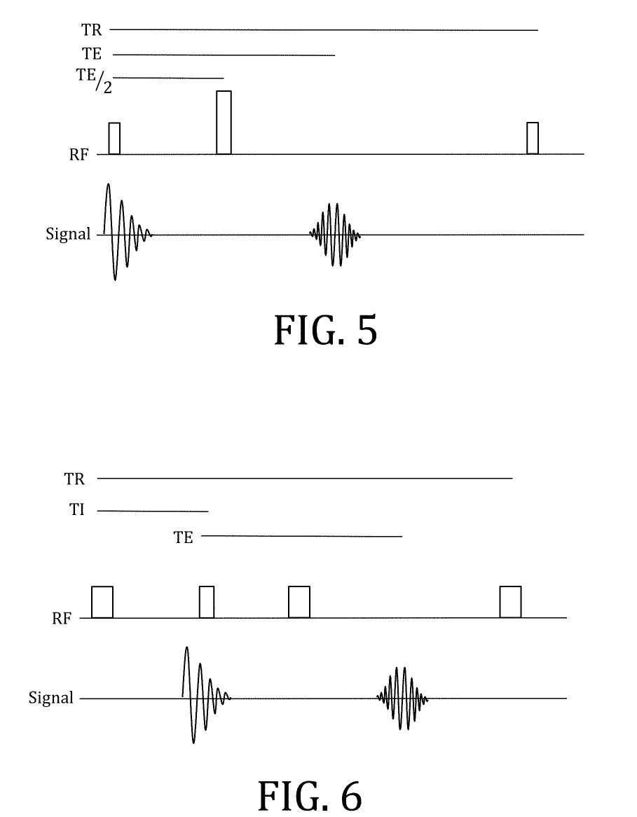

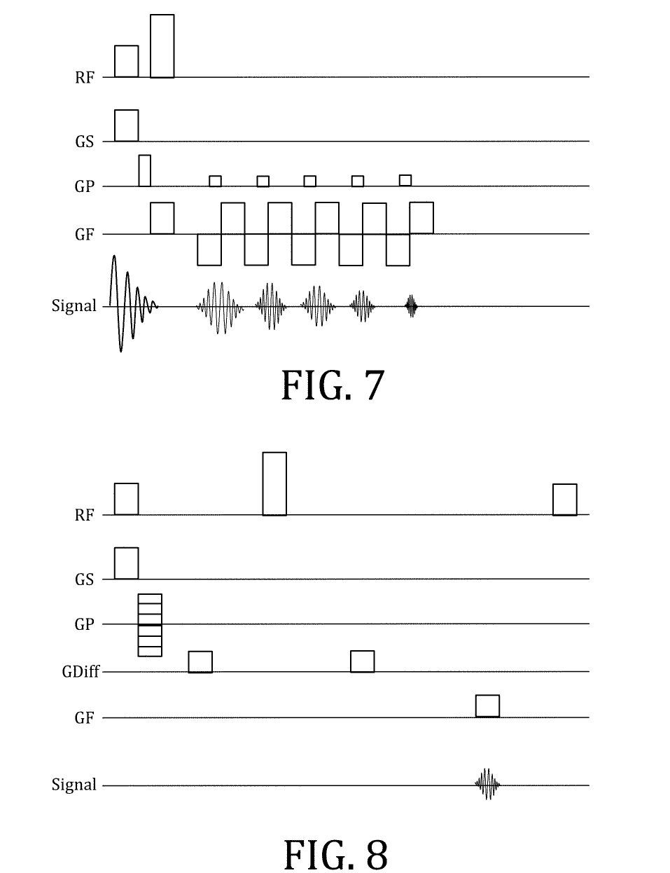

The relaxation times and proton density may be estimated from MRI using one or more pulse sequences whereby a series of radiofrequency pulses, magnetic gradient pulses, or both are applied in a regular pattern to perturb protons of water in a known manner. Exemplary pulse sequences may include spin echo (“SE”) for estimating T1, T2, and proton density; echo planar imaging (“EPI”) for estimating diffusion tensor. Exemplary SE sequences (conventional (FIG. 5) and inversion recovery (FIG. 6)) and diffusion sequences (EPI (FIG. 7) and diffusion-weighted (FIG. 8)) are shown. One of ordinary skill in the art having the benefit of the disclosure provided herein would understand selection of pulse sequence parameters, including radio frequency (“RF”) power, echo times (“TEs”), repetition times (“TRs”), inversion times (“Tis”), gradient strength (for slice, phase, and so forth), phase, and diffusion gradients (GDiff), for example.

For example in FIG. 2, extracellular fluid (or specifically, extra-axonal fluid) may be estimated according to one embodiment of the present invention by using a three pool model. The three-pool model uses Mill signal to estimate fractions of white matter signal originating from three populations of water protons: water inside myelinated axons (ma), water trapped in myelin of axons (my), and the water outside myelinated axons as the mixed fraction (mx). An important factor in the three-pool model is the transfer of water between the three compartments: ma, my, and mx. It is conventionally accepted that intra-axonal water exchange behaves as a fast process as compared to T1 relaxation but as a slow process as compared to T2 relaxation. Thus, the three pool model presumes that protons tightly bound in myelin 152 of axons 145 have different relaxation characteristics than protons associated with cytoskeletal components (not shown) within the axon 146 or protons loosely bound in interstitial fluid.

The three-pool model is formulated as follows:

f_{my}+fₘₐ+fₘₓ=1 Equation 8,

where fₓₓ is a fraction of signal coming from pool xx. Equation 8 indicates that the entirety of the signal comes from one of the three compartments (axon, myelin, and extra-axonal, respectively).

The three-pool model further includes:

f_{my}w1_{my}+fₘₐw1ₘₐ+fₘₓw1ₘₓ=1 Equation 9,

where w1ₓₓ is a fractional relaxation rate from pool xx and w1ₓₓ=R1ₓₓ/R1. R1 is a measured relaxation rate, where R1=1/T1. R1ₓₓ is the relaxation rate (1/Tₓₓ) for pool xx. Equation 9 indicates a linear combination of relaxation rates from each of the three pools is responsible for a total relaxation rate, which is a consequence of fast exchange processes where the pools become mixed within the T1 relaxation time.

The three-pool model further includes:

$${S}_{PDW} = S ( 0 ) [ {f}_{my} {e}^{- ( \frac{TE 1}{T {2}_{my}} )} + {f}_{ma} {e}^{- ( \frac{TE 1}{T {2}_{ma}} )} + {f}_{mx} {e}^{- ( \frac{TE 1}{T {2}_{mx}} )} ] \\ {S}_{T 2 W} = S ( 0 ) [ {f}_{my} {e}^{- ( \frac{TE 2}{T {2}_{my}} )} + {f}_{ma} {e}^{- ( \frac{TE 2}{T {2}_{ma}} )} + {f}_{mx} {e}^{- ( \frac{TE 2}{T {2}_{mx}} )} ] , \qquad Equation 10$$

where S_{PDW} and S_{T2W} are PD and T2-weighted signals from an acquired image, fₓₓ is the pool fraction, TE1 and TE2 are echo times of the PD and T2 pulse sequences, respectively, and T2ₓₓ are T2 values of the individual pools. From the ratio of these two equations, and by defining w2ₓₓ:

$$\begin{matrix}w {2}_{xx} = \frac{{S}_{PDW}}{{S}_{T 2 W}} * {e}^{- ( \frac{TE 2}{T {2}_{xx}} )} - {e}^{- ( \frac{TE 1}{T {2}_{xx}} )} , and & Equation 11 \\ {f}_{my} w {2}_{my} + {f}_{ma} w {2}_{ma} + {f}_{mx} w {2}_{mx} = 0. & Equation 12\end{matrix}$$

Equation 12 represents T2 behavior as a multi-exponential, where residence time of spins in each pool is long compared to the relaxation time. As such, each pool exhibits independent exponential relaxation.

Equations 7, 9, and 12 may then be solved for each pool fraction, f_{my}, fₘₐ, and fₘₓ:

$$[ \begin{matrix}{f}_{my} \\ {f}_{ma} \\ {f}_{mx}\end{matrix} ] = {[ \begin{matrix}1 & 1 & 1 \\ w {1}_{my} & w {1}_{ma} & w {1}_{mx} \\ w {2}_{my} & w {2}_{ma} & w {2}_{mx}\end{matrix} ]}^{- 1} [ \begin{matrix}1 \\ 1 \\ 0\end{matrix} ] . \qquad Equation 13$$

The weight factors in Equations 7, 9, and 12 are dependent on the several measures that may be directly calculated from MRI images: w1s may be derived from T1 values (such as by a series of inversion recovery images), and w2s may be derived from T2 and PD images. The w1s and w2s may also be a function of T1 of each pool (T1ₓₓ) and T2 of each pool (T2ₓₓ), which must be assigned before solving for the water fractions using Equation 13.

In view of the foregoing, and at start with reference still to FIG. 4, images may be acquired on any suitable MRI instrument configured to operate the SE and EPI pulse sequences and at any field strength, with the caveat that a suitable signal-to-noise ratio be attainable for particular regions of interest (for example, the corpus callosum 116 (FIG. 1)). Imaged anatomy or phantom may be in vivo or in situ. For example, images may be acquired using a Tim Trio 3 Tesla scanner (Siemens Medical Solutions USA, Inc., Malvern, Pa.) using an appropriate RF coil, such as an extremity coil or head coil. While the number and type of images acquired may vary, generally T1-, T2-, PD, and DWI may be acquired (Block 172).

Before image processing, a region of interest (“ROI”) may be determined, wherein each ROI may comprise one or more voxels (Block 174). If comprising more than one voxel, the ROI may be manually or automatically selected. Generally, ROIs of more than one voxel comprise a single anatomical feature; however, the use of ROIs should not be so limiting.

With images acquired and ROI determined, a magnitude of conduction of extra-axonal fraction within one or more ROIs may be determined using Equation 13, above (Block 176).

Referring again to FIG. 4, and with extra-axonal fluid fraction determined, longitudinal and transverse directions of conductivity may be determined (Block 178). In that regard, signal values from the inversion recovery images may be fit to a model to determine approximate T1 value according to:

$$S = {S}_{0} ( 1 - 2 {e}^{\frac{TI}{T 1}} ) , \qquad Equation 14$$

where S is the signal from the image voxel, S₀ and T1 are fitting parameters, and TI is the inversion recovery time from the image sequence. Signal values from the T2 weighted and PD images may be used to calculate the T2 value according to:

$$S = {S}_{0} {e}^{\frac{TE}{T 2}} . \qquad Equation 15$$

Diffusion images may be used to visualize the diffusion direction and to determine a direction of the anisotropy in conductivity. Diffusion tensors for each voxel may be visualized such that diffusion data cloud plots consisting of the diffusion coefficient in each of the 64 diffusion directions based on:

S=S₀e^{-bD} Equation 16,

where S is the signal from the voxel in the diffusion image, S₀ is the signal from the voxel in the non-diffusion (b=0) image, and b is the degree of diffusion weighting. According to some embodiments, b of 1000 may be used for the diffusion images; however, other values of b may alternatively be used. D is the apparent diffusion coefficient, a measure of the degree of diffusion in the direction of the diffusion gradient. The diffusion tensor may be described as:

$$D = [ \begin{matrix}{D}_{xx} & {D}_{xy} & {x}_{xz} \\ {D}_{yx} & {D}_{yy} & {D}_{yz} \\ {D}_{zx} & {D}_{zy} & {D}_{zz}\end{matrix} ] . \qquad Equation 17$$

According to one embodiment of the present invention, eigenvectors and eigenvalues of D may be derived in accordance with T. E. J. BEHRENS et al., “Characterization and propagation of uncertainty in diffusion-weighted MR Imaging,” MRM, Vol. 50 (2003) 1077-1088, the disclosure made therein being incorporated herein by reference, in its entirety. Such methodology may be implemented by way of the Oxford Center for Functional MRI of the Brain (“FMRIB”) Software Library (“FSL”), available at https://fsl.fmrib.ox.ac.uk/fsl/fslwiki/FSL. However, other methods of determining angular components of diffusion (direction and magnitude in respective directions) may also be used.

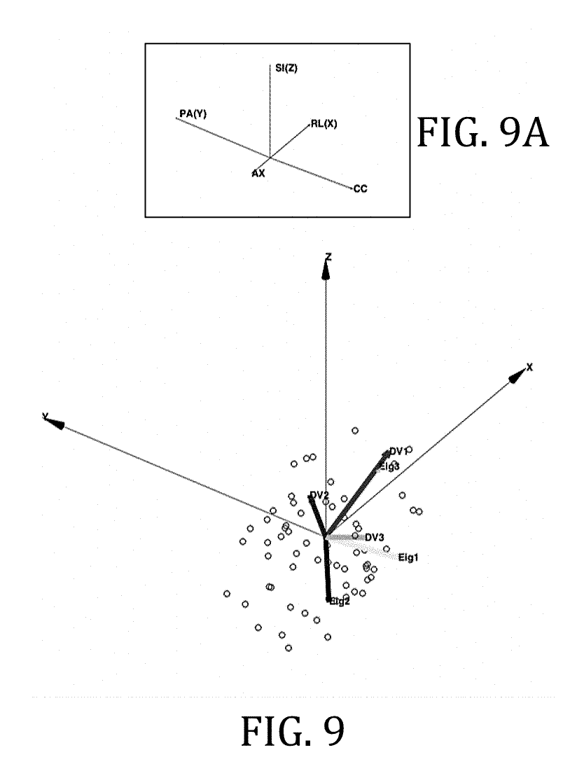

According to one embodiment, plots of the diffusion eigenvectors weighted by the diffusion eigenvalues may be calculated, as illustrated in FIG. 9. In this way, diffusion images may be used to visualize the diffusion direction and to determine the direction of the anisotropy in the white matter conductivity model. A diffusion tensor of the ROI may be visualized, such as to a MATLAB code, by plotting diffusion data cloud (circles) consisting of the diffusion coefficient in each of the 64 diffusion directions (Equation 16).

Continuing with FIG. 9, and along with the data cloud, weighted diffusion eigenvectors may be plotted by the diffusion eigenvalues (illustrated as “DV1,” “DV2,” and “DV3”). Eigenvectors/eigenvalues of the diffusion data cloud may be computed by principle components analysis (illustrated as “Eig1,” “Eig2,” “Eig3”).

In the illustrated example of FIG. 9, the reference axes (the small axes at the top of the figure) from the T2 image, superior/inferior (SI) corresponding to the Z axis of the MRI magnet, posterior/anterior (PA) corresponding to the Y axis of the magnet, and right/left (RL) corresponding to the X axis of the magnet, as well as the AP axis of the corpus callosum, and a secondary axis (AX) corresponding to mirror image points on the right and left of the frontal lobes (showing the brain's tilt as placed in the imager). The alignment axes allow visualization of the orientation of the brain with the scanner axes.

According to some embodiments, the plots may be rotatable to enable visualization of the vectors in all directions. For example, in FIG. 9, the longest eigenvector (eig3) and the longest diffusion vector (DV1) are aligned and are very close to the x-axis. The transverse vectors (eig1, eig2, DV2, and DV3) are fairly closely aligned with each other and align with the y and z-axes.

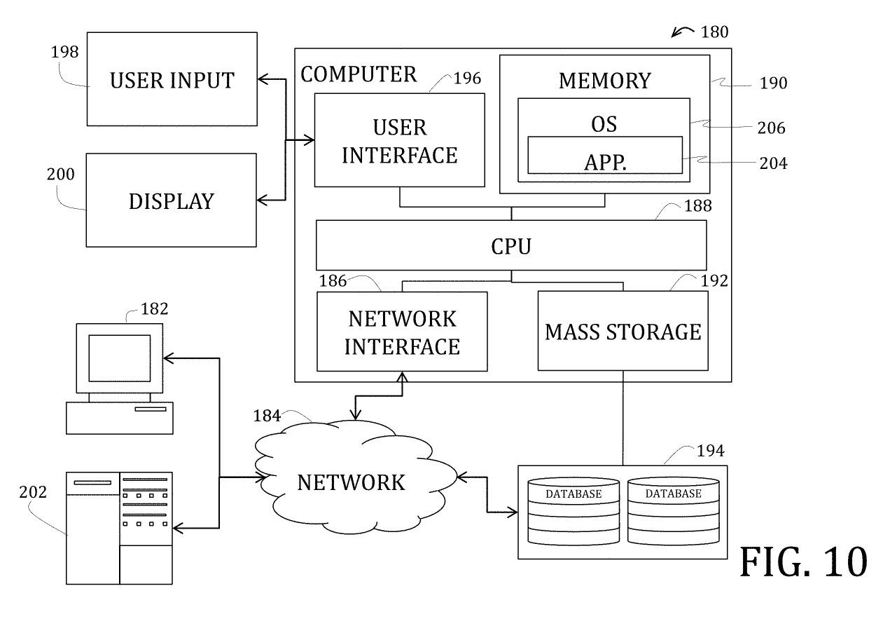

The foregoing, and in particular the methods of FIG. 4, may be preferably accomplished by a computer 180, which is described in greater detail with respect to FIG. 10 and which may be considered to represent any type of computer, computer system, computing system, server, disk array, or programmable device such as multi-user computers, single-user computers, handheld devices, networked devices, or embedded devices, etc. The computer 180 may be implemented with one or more networked computers 182 using one or more networks 184, e.g., in a cluster or other distributed computing system through a network interface 186 (illustrated as “NETWORK I/F”). The computer 180 will be referred to as “computer” for brevity's sake, although it should be appreciated that the term “computing system” may also include other suitable programmable electronic devices consistent with embodiments of the invention.

The computer 180 typically includes at least one central processing unit 188 (illustrated as “CPU”) coupled to a memory 190 along with several different types of peripheral devices, e.g., a mass storage device 192 with one or more databases 194, an input/output interface 196 (illustrated as “USER INTERFACE” with associated display 198 and user input device 200), and the Network I/F 186. The memory 190 may include dynamic random access memory (“DRAM”), static random access memory (“SRAM”), non-volatile random access memory (“NVRAM”), persistent memory, flash memory, at least one hard disk drive, and/or another digital storage medium. The mass storage device 192 is typically at least one hard disk drive and may be located externally to the computer 180, such as in a separate enclosure or in one or more networked computers 182, one or more networked storage devices (including, for example, a tape or optical drive), and/or one or more other networked devices (including, for example, a server 202).

The CPU 188 may be, in various embodiments, a single-thread, multi-threaded, multi-core, and/or multi-element processing unit (not shown) as is well known in the art. In alternative embodiments, the computer 180 may include a plurality of processing units that may include single-thread processing units, multi-threaded processing units, multi-core processing units, multi-element processing units, and/or combinations thereof as is well known in the art. Similarly, the memory 190 may include one or more levels of data, instruction, and/or combination caches, with caches serving the individual processing unit or multiple processing units (not shown) as is well known in the art.

The memory 190 of the computer 180 may include one or more applications 204 (illustrated as “APP.”), or other software program, which are configured to execute in combination with the Operating System 206 (illustrated as “OS”) and automatically perform tasks necessary for operating the transducers and/or reconstructing the images with or without accessing further information or data from the database(s) 194 of the mass storage device 192.

Those skilled in the art will recognize that the environment illustrated in FIG. 10 is not intended to limit the present invention. Indeed, those skilled in the art will recognize that other alternative hardware and/or software environments may be used without departing from the scope of the invention.

The following examples illustrate particular properties and advantages of some of the embodiments of the present invention. Furthermore, these are examples of reduction to practice of the present invention and confirmation that the principles described in the present invention are therefore valid but should not be construed as in any way limiting the scope of the invention.

### Example 1—Calibrating the Three Pool Model

The three pool model according to the illustrative embodiment of FIG. 4, was calibrated for MRI having B₀ of 1.9 T by scanning human subjects aged from infancy to teenagers and using T1 and T2ₓₓ values from literature. Calibration of the three pool model was necessary to evaluate images of pig subjects at field strengths of 3 T.

Two complicating factors are presented: B₀ change from 1.9 T to 3 T (significantly altering T1 values) and the use of pig brains (born with significant myelination as compared to humans). T1 values at varying field strengths may be estimated by:

T₁=0.583(B₀)^{0.382} Equation 18.

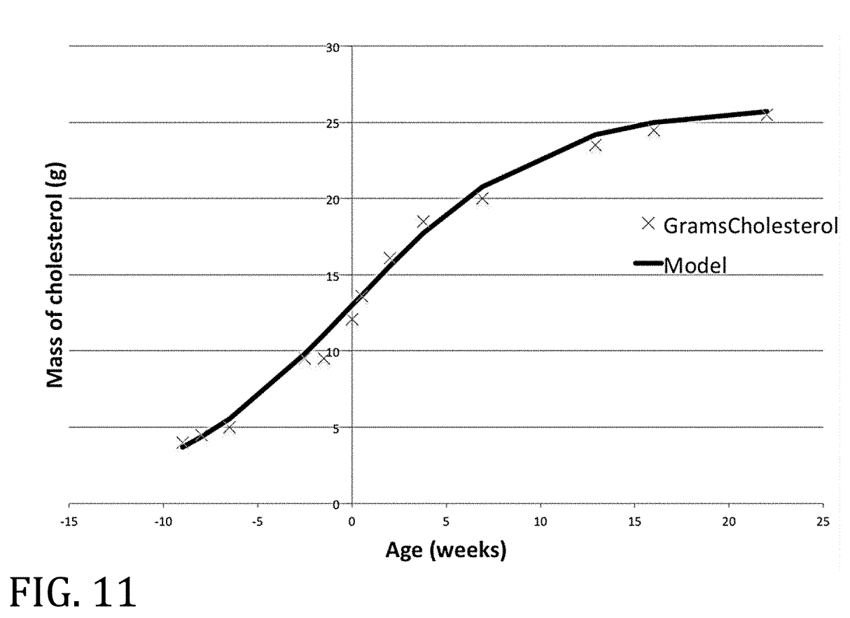

The mass of cholesterol in brains of pigs at various ages has been measured previously. Normalized pool fraction values as a function of time were modeled by a nonlinear fit to the functions:

$$\begin{matrix}{f}_{my} = \frac{{f}_{myss}}{1 + {e}^{- ( {b}_{3} * age )}} ; & Equation 19 \\ {f}_{my} = \frac{{f}_{mass}}{1 + {e}^{- ( {b}_{2} * age )}} ; and & Equation 20 \\ {f}_{my} = \frac{{f}_{mxss}}{1 + {e}^{- ( {b}_{1} * age )}} , & Equation 21\end{matrix}$$

where f_{myss}, fₘₐₛₛ, and fₘₓₛₛ are steady state (fully myelinated) pool fractions; b₁, b₂, and b₃ are rate constants; and age is measured in weeks. FIG. 11 illustrates mass of brain cholesterol in pigs as a function of age. Reasonable starting values are provided in Table 1:

TABLE 1

| Variable | Value Range (ms) |
|---|---|
| T1_{my} | 400-600 |
| T1ₘₐ | 900-1200 |
| T1ₘₓ | 1500-3500 |
| T2_{my} | 8-12 |
| T2ₘₐ | 36-44 |
| T2ₘₓ | 110-150 |

As a target for model calibration, sagittal slices of brain tissue were stained for myelin and compared to a human corpus callosum control. Human corpus callosum genu values were assumed to apply to the human corpus callosum slices, and a comparative color thresholding technique was used to find an estimate of the pig corpus callosum genu myelinated axon fraction (fₘₐ). The myelin fraction (f_{my}) would be expected to increase with the same characteristic as the ingrowth of cholesterol, so the knee of the myelin in-growth curve was targeted to match the knee of the myelin fraction curve. Finally, the values of the model parameters were manipulated to make all pool fractions positive and to reduce the standard deviation of the modeled steady state pool fractions to a minimum.

Yorkshire pigs and Yucatan minipigs, aged 15 and 104 weeks were obtained at time of euthanasia. Additionally, a stillborn Yorkshire pig was obtained from the same farm that supplies the research subjects (age 0 weeks). Brains were removed by removal of the top of the skull and placed in a solution of 0.9% veterinary saline for up to 1 hr. The brains were then encapsulated in a 4 in diameter sphere of 4% agarose for subsequent imaging. Images were acquired according to the sequences and parameters provided in Table 2, below.

TABLE 2

| | | GEOMETRIC | |
|---|---|---|---|
| SEQUENCE | PARAMETERS | PARAMETERS | PURPOSE |
| SE, T1-weighted | TR = 1100 ms | Voxel Size: 1 × 1 × 1.8 mm | Geometric reference, |
| | TE = 13 ms | Matrix: 256 × 192 | Calculate T2, Three- |
| | NA = 1 | Slice Thickness: 1.5 mm | pool model |
| SE, T2-weighted | TR = 5000 ms | | |
| | TE = 80 ms | | |
| | NA = 1 | | |
| SE, Proton | TR = 5000 ms | | |
| Density- | TE = 13 | | |
| weighted | NA = 1 | | |
| Fast Low Angle | TR = 10,000 ms | Voxel Size: | Calculate T1 |
| Shot (FLASH) | TE = 1.9 ms | 1.5 × 1.5 × 4.55 mm | |
| Inversion | NA = 3 | Matrix: 128 × 96 | |
| Recovery | FA = 7 | Slice Thickness: 3.5 mm | |
| Sequences | TI = 200, 500, 700, | | |
| | 900, 1500, 2000, and | | |
| | 3000 ms | | |

T1 was calculated as:

$$S ( T 1 ) = [ 1 - 2 {e}^{- ( \frac{TI}{T 1} )} ] . \qquad Equation 22$$

A second group of pig brains (Yorkshires aged about 15 weeks) were extracted as described above but fixed with 10% neutral buffered formalin solution for several days. The corpus callosum of each brain was excised and encased in paraffin for histology slicing.

Slices were stained with Luxol Fast Blue (“LFB”), Hematoxylin and Eosin (“H&E”), and Periodic Acid Schiff (“PAS”) stains. The LFB stains myelin and the other stains are used to differentiate other parts of cells. This combination is routinely used to detect demyelination.

Table 3, below, is the estimated myelinated axon fraction (fₘₐ) for several different locations on the control image and the pig images.

TABLE 3
MYELINATED AXON FRACTION

| | Human (controls) | Pig |
|---|---|---|
| | 0.55 | 0.58 |
| | 0.51 | 0.46 |
| | 0.50 | 0.47 |
| | 0.64 | 0.52 |
| | 0.63 | 0.59 |
| | 0.48 | 0.55 |
| Avg | 0.55 | 0.53 |
| Std Dev | 0.068 | 0.055 |

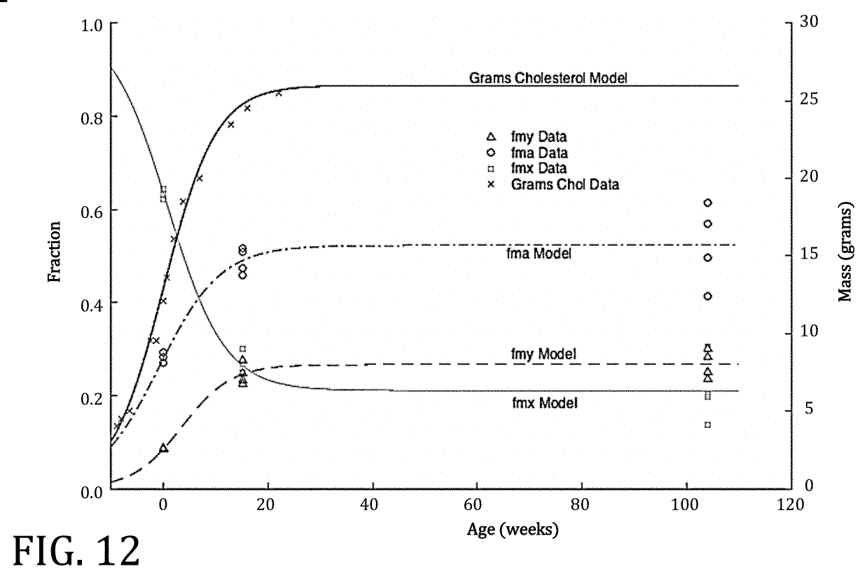

Based on the model calibration criteria, the calibrated model parameters are in FIG. 12 and Table 4, below. This set of parameters meets the criteria of having an fₘₐ close to the value estimated from histology, the knee of the f_{my} curve located at the same location as the knee of the cholesterol in-growth curve, and the variability of the steady state pool fractions a minimum. These values will be the basis of conductivity predictions in white matter.

The method described herein could be used to calibrate the three-pool model for other species and at other main magnetic field strengths. Histoanatomical methods could be refined to be more specific for myelin to the exclusion of glial cells and to use identical brain slicing, fixation, and staining techniques to reduce the need for color correction between the sample and the controls. But the values of the mixed fraction achieved by the calibration have a standard deviation of approximately 10% (Table 5).

TABLE 4

| Parameter | Value (mS) |
|---|---|
| T1_{my} | 349 |
| T1ₘₐ | 1620 |
| T1ₘₓ | 2835 |
| T2_{my} | 10 |
| T2ₘₐ | 40 |
| T2ₘₓ | 130 |

TABLE 5
STEADY STATE POOL FRACTIONS

| Pool | Value | Stnd Dev. |
|---|---|---|
| f_{my} | 0.266 | 0.0114 |
| fₘₐ | 0.522 | 0.0284 |
| fₘₓ | 0.211 | 0.0230 |

### Example 2—Conductivity by MRI Model

Recently sacrificed Yucatan minipigs and Yorkshire pigs, between 3 months and 2 years old, were obtained through a tissue sharing protocol. The brains were removed from the pigs within 30 min of sacrifice by removal of top of skull and dura to expose the top of the cerebrum. Brains were carefully handled to retain shape and placed in 0.9% veterinary saline (σ≅17 mS/cm). After approximately 1 hr, the brains were encapsulated in 4% agarose and imaged.

Images were collected using the Siemens Tim Trio 3 Tesla scanner and an extremity coil. Pig brains were encapsulated in a 4 in sphere of 4% agarose to minimize susceptibility artifacts and geometric distortions from non-uniform magnetic fields. The imaging sequences collected are shown in Table 2, above.

Images were viewed and processed in Mango by importing from the DICOM format and saved as NIFTI files. Images with identical geometric parameters were stacked as a time series so processing steps could be applied to all images simultaneously. For instance, all the inversion recovery images were stacked in order of increasing inversion time (T1) so signal strength can be analyzed versus T1 for the purpose of estimating spin-lattice relaxation time (T1). The diffusion-weighted EPI images (“DWI”) were processed using software tools from Oxford Center for FMRIB. The tools, FSL, FSL Diffusion Toolbox (FDT) were used to transform the diffusion images from the MRI scanner into DTI and several other useful metrics. UTHSCSA'S (University of Texas Health Science Center at San Antonio) MANGO software was used for image processing and registration.

DWIs came from the image server as a stack of 65 individual images. The first image of the stack had no diffusion weighting (b=0); subsequent images were weighted for each of 64 diffusion-weighting directions and strengths. In that regard, the diffusion-weighting was achieved by applying a very large gradient field in one polarity, then applying a gradient of equal strength with opposite polarity at some time later. The first diffusion gradient dephases the spins within the weighted volume and the second (equal and opposite) gradient rephrases the spins within the same weighted volume. Spins within the weighted volume having a location or phase that is different from before the gradients were applied are not properly rephased and do not produce signal. Therefore, a pixel signal is decreased by a proportional amount of spins that have diffused out of the voxel.

The direction of the diffusion gradients was varied to provide 64 non-collinear diffusion directions. Eddy currents, when present, were corrected with Eddycorrect, which applies an affine transform (12 degrees of freedom; three translations, three rotations, three scalings, and three skews) to each diffusion image with reference to the non-diffusion-weighted (b=0) image, e.g., the first image of the stack. The resulting set of images is well registered and can be used for diffusion tensor image processing.

Eddy current corrected images, or images with no need of Eddy current correction, were processed with FDT DTIFIT using a 3-dimensional Gaussian fit routine. In this way, the signal from each diffusion direction is used to derive eigenvectors and eigenvalues of the diffusion direction ellipse.

Inputs to the FDT code are the diffusion image, the b-vectors and b-values extracted from the DICOM image headers, and a mask of the non-diffusion (b=0) image. Outputs of FDT include image matrices that assign a value of a parameter to each voxel. Parameters with multiple values are output as stacked images (for instance, eigenvectors are output as three stacked images with the x-, y-, and z-components for each voxel in succeeding images). A single image was output for each eigenvalue (these are subsequently stacked), Fractional Anisotropy (“FA”), and Mean Diffusivity (“MD”). FSL assigned a parameter, M0, to each diffusion ellipse signifying the overall ellipse shape (when M0 is 0, the ellipse is a sphere). M0 approaching unity indicates a prolate spheroid, an ellipse that has one eigenvector much longer than the other two, and M0 approaching negative one indicates and oblate spheroid, or disk shape where one of the eigenvectors are much smaller than the other two.

### Example 3—Conductivity by 4EM

After imaging as provided in Example 2, the top of the agarose sphere were carefully removed to reveal a top portion of the brain tissue. A landmark was selected that is recognizable on T2 MRI and on the physical brain (usually the highest point on the frontal or parietal lobe) as a reference for measurement.

Based on the FA image, two locations along the midline of the corpus callosum were selected as having the highest FA and were used as targets for measurement. The 4EM system electrodes are fixed in a Huxley style micropositioner (Sutter Instruments Co., Novato, Calif.). The electrode tips were zeroed to the reference location, which became the depth reference plane. The brain was oriented to match the images by depth reference measurement at several locations around the upper lobe.

The location of each measurement was measured along the longitudinal fissure from the location of the reference point and marked with India ink. The probes of the 4EM system are inserted into the tissue, aligned with the longitudinal fissure, and moved downwardly 1 mm at a time while measuring the conductivity at each step through the depth of the corpus callosum. Once the probes were moved past the depth of the corpus callosum, the probes are withdrawn and rotated 90° to the longitudinal fissure and reinserted in 1 mm increments, again measuring conductivity at each step. The axons in the corpus callosum are presumed to run primarily along a lateral direction; therefore, the potential measurements across the longitudinal fissure are ϕ_{L} and the measurements along the longitudinal fissure are ϕ_{T}.

The probes include four electrodes: two lower impedance electrodes are used to deliver a current to the medium and two higher impedance electrodes are used to measure the potential in the medium. The higher impedance electrodes draw a minimal amount of current, minimizing electrolytic interactions and polarization effects at the electrode/electrolyte interface.

The response of the 4EM in homogeneous, isotropic media has been described:

$$V = \frac{I}{4 π σ a} , \qquad Equation 23$$

where the electrodes are aligned linearly with the two high impedance potential measurement electrodes (V1, V2) and flanked by the two current injection electrodes (I1, I2). Equation 23 assumes electrodes have uniform spacing, a, and are surrounding by a medium of uniform conductivity, σ.

Infinite half space of anisotropic material, longitudinal and transverse to fiber direction:

$$\begin{matrix}{V}_{si , longitudinal} = \frac{1}{2 π a {σ}_{transverse}} , and & Equation 24 \\ {V}_{si , transfers} = \frac{1}{2 π a} \sqrt{\frac{1}{{σ}_{transverse} * {σ}_{longitudinal}}} . & Equation 25\end{matrix}$$

Equation 23 provides a solution for an infinite space with isotropic conductivity, while Equations 24 and 25 provide solutions for electrodes on the surface of an anisotropic half-space. Equations 24 and 25 may be modified to predict the response in an infinite anisotropic space by multiplying by a factor of ½. As long as the distance between the outer electrodes and the boundary are more than approximately three times the electrode spacing (3*a) the boundary will have minimal impact on the measurement.

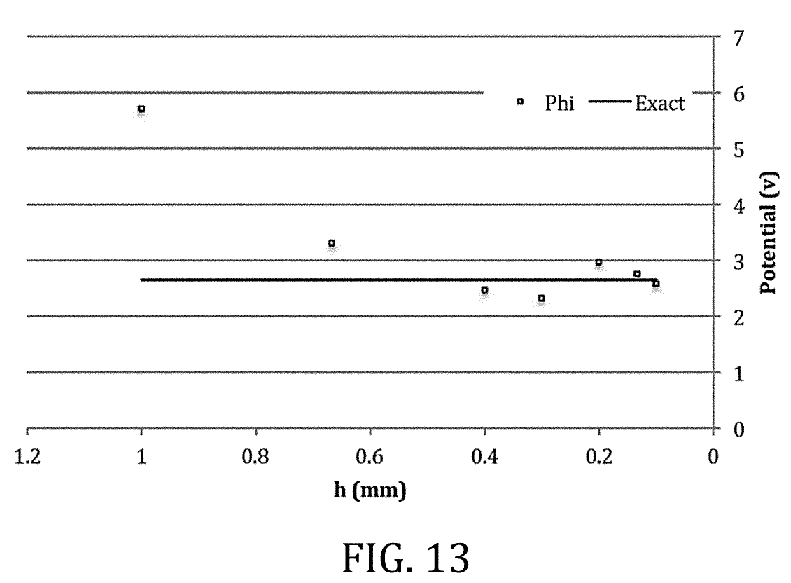

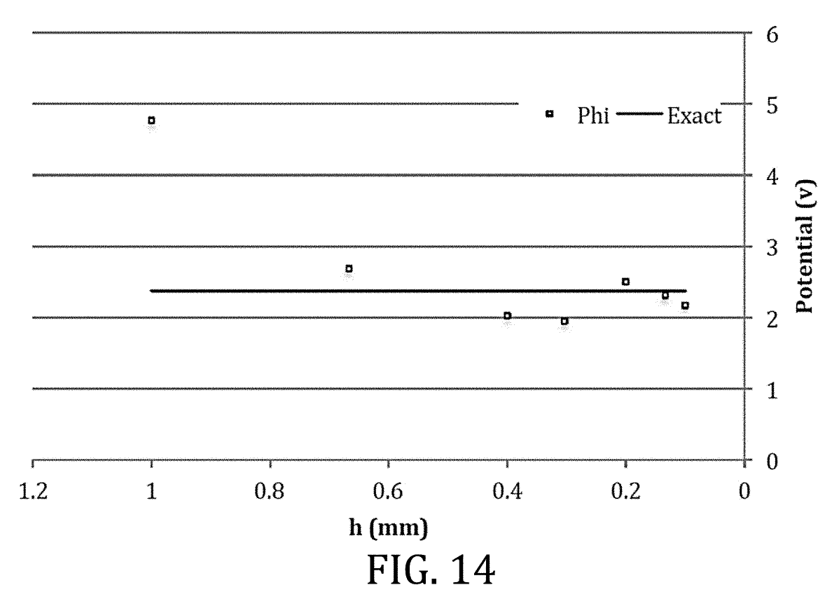

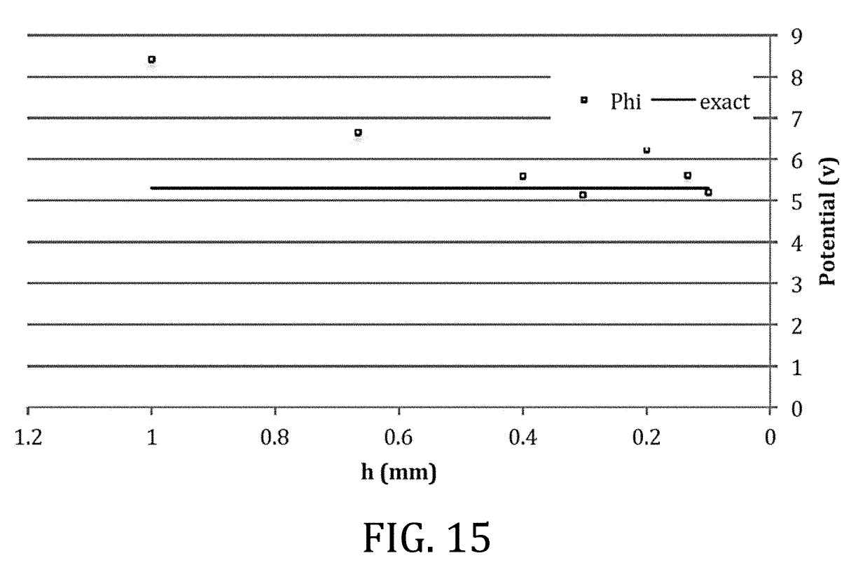

Finite difference models are validated based on convergence to the known exact value for the solution with decreasing voxel size (h). FIG. 13 is a graph of predicted potential in an isotropic infinite space with decreasing voxel size as compared to the exact solution from Equation 23. FIGS. 14 and 15 are graphs of predicted potential versus voxel size for longitudinal and transverse probe orientations in an infinite anisotropic space (σₗ=0.5; σₜ=0.1). FIGS. 13-15 demonstrate fairly close convergence to the analytical solution with decreasing voxel size (approximately 8% difference for the anisotropic transverse cases and approximately 2% for the other cases).

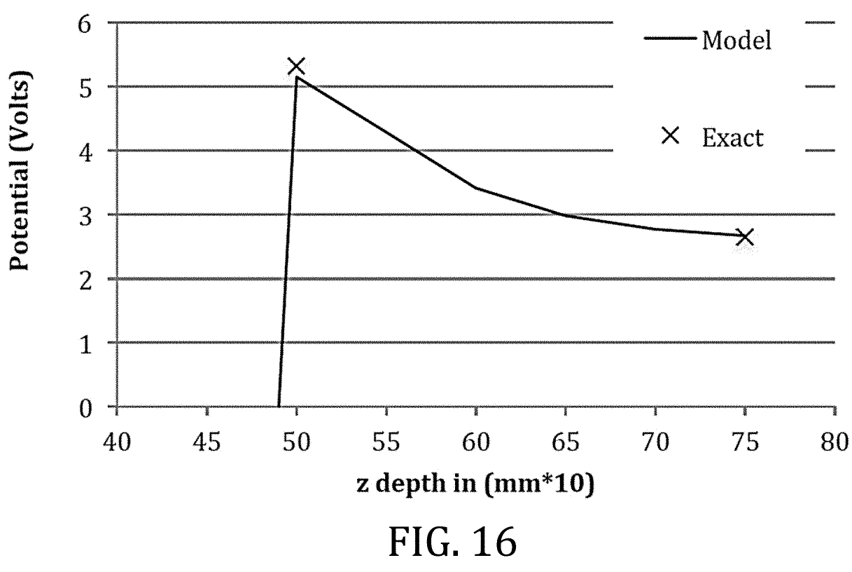

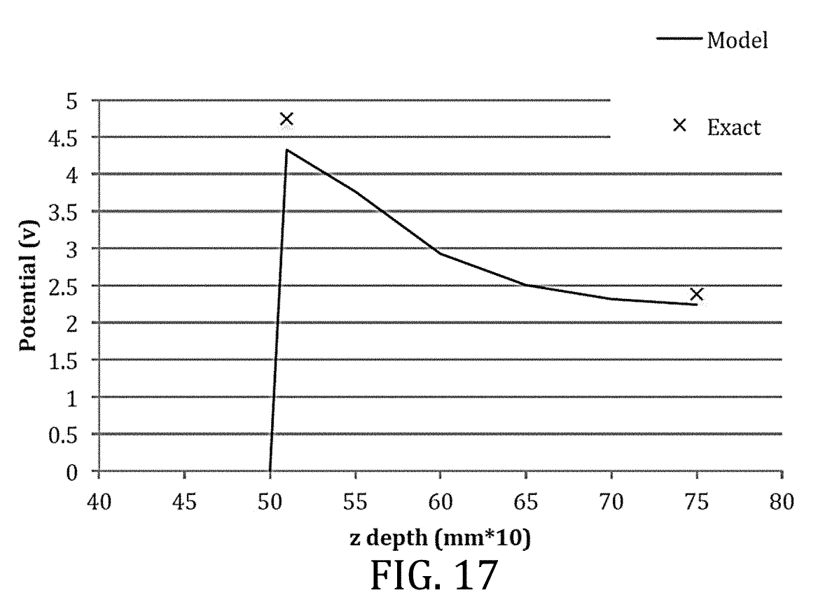

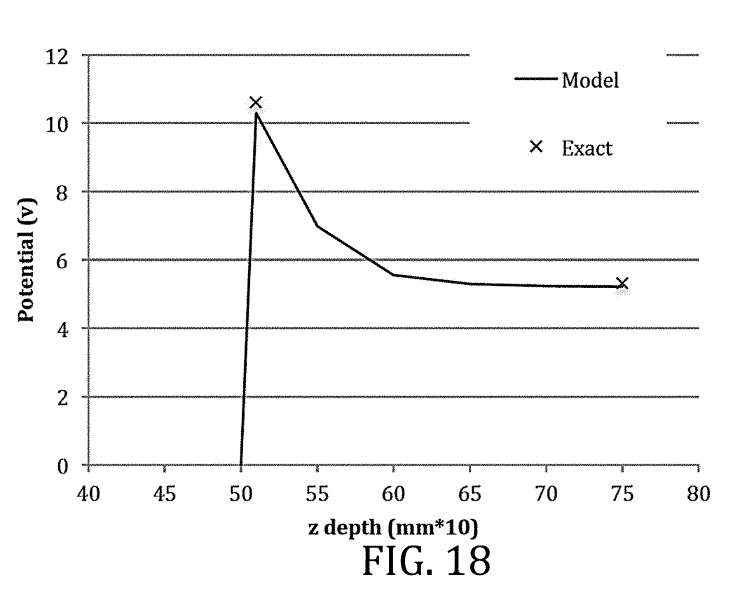

Models may be further validated by comparing them to theory over a range of values. FIGS. 16-18 compare model predictions at increasing depth with theoretical predictions (Equations 24 and 25) at the surface of an infinite half space and at depth (essentially an infinite space). The finite difference model slightly under predicts (by about 8%) the value at the interface, which may be caused by the simplistic formulation (kernel) of the finite difference model. However, at depth, the model predicts within approximately 5% of the theoretical value.

Accepting that the finite difference model is a fairly good predictor of the response of the 4EM, a number of configurations can be examined. In use, the 4EM is advanced down through tissue through a layer that is thought to be anisotropic. In order to understand the response as the probes traverse the anisotropic layer, a geometry model was built with an anisotropic layer, 2 mm in thickness, flanked by infinite isotropic layers. Isotropic infinite space and isotropic infinite half-space were also analyzed.

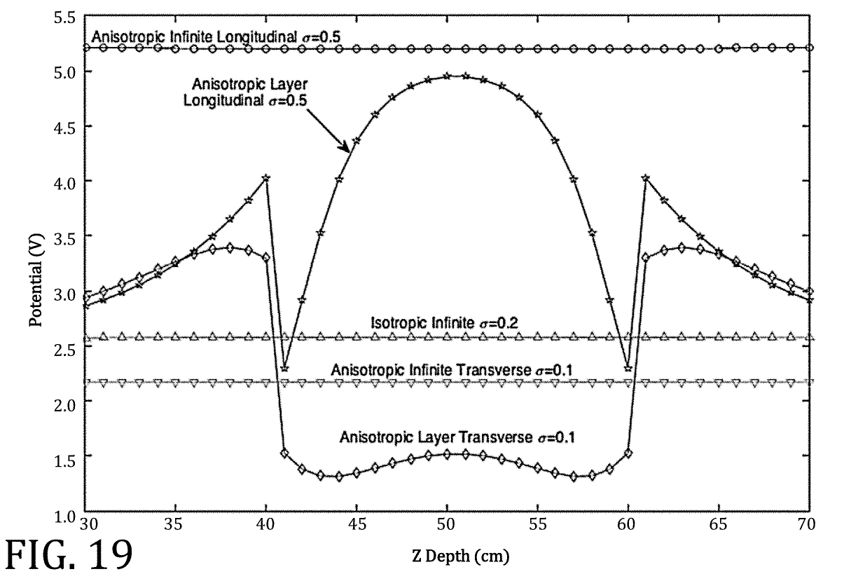

FIG. 19 shows the predicted response of the 4EM system as the probe is traversed from 3 mm to 7 mm in depth (the z direction) in the center (x and y directions) of the space. The isotropic media in these analyses have an electric conductivity of σ=0.2 mS/cm and the anisotropic media have conductivities longitudinal σₗ=0.5 mS/cm, and transverse σₜ=0.1 mS/cm.

The response of the 4EM system probes in the anisotropic layer is reduced by interactions with the surrounding medium as compared to the infinite case. In the transverse direction, the response is reduced by 30.2% and in the longitudinal direction the response is reduced by 20.2%.

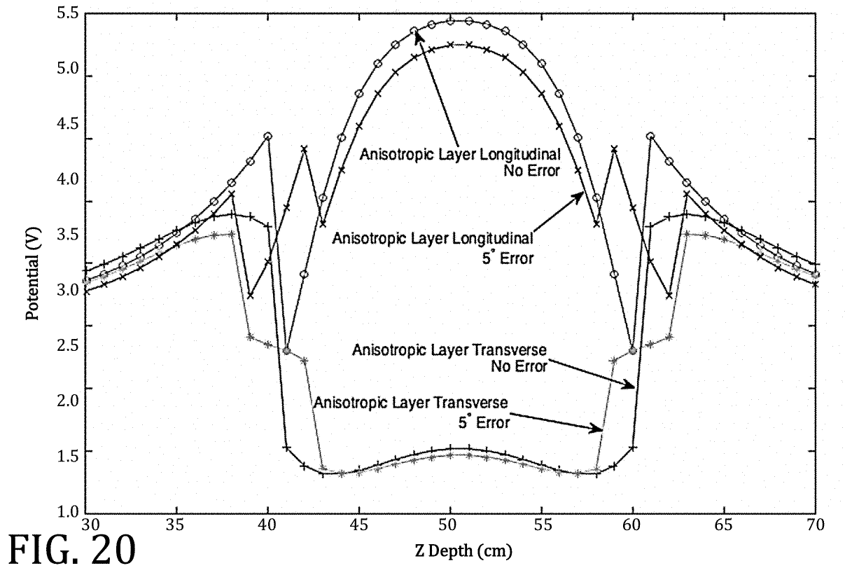

Another use of the finite difference model is to explore the impact of probe positioning error on the 4EM response. The probes enter the layer at different probe depths, z. The effect of may be seen in the graphs of FIG. 20. The error causes the response of the 4EM system to be reduced by 4% in the longitudinal direction and 3.3% in the transverse direction.

The FD model converged to the closed form result and agreed with the closed form solutions across a range of values within 10% in all cases. Furthermore, the predicted response of the 4EM system is reasonable as the probes traverse down through an anisotropic layer. Future work on this effort could include measurement of anisotropic media of known conductivity to further validate the model.

The FD model will be extremely useful to those who need to directly measure conductivity with depth in tissue if the nature of the tissue structure is known well enough to form a geometric model. It is also useful to analyze a range of tissue and geometric properties (e.g., layer thickness, anisotropy ratio) for comparison to measured data and to explain 4EM system response in anisotropic heterogeneous media.

### Example 4—Calibration and Correction of 4EM

The 4EM system was calibrated using a series of standard electrolyte solutions with known conductivities measured using a NIST traceable conductivity meter. The solutions are an isotropic medium so the 4EM response to immersion in the calibration solutions is described by Equation 23.

Equations 24, 25, and Ohm's law (J=σE) were for a point current source and sink, an idealized case that could not be realized. Two major effects of immersing physical electrodes to various depths must be accounted for: 1) a change in the shunt capacitance proportional to depth that divides the current injected into the system, and 2) the effect of boundaries of the medium and regions of heterogeneous and anisotropic conductivity that affect the response of the system. Accurate estimates of conductivity must therefore incorporate corrections for each of these.

The shunt capacitance of an electrode may be found using the equation C=Q/V, where Q is the charge on a conductor (therefore, −Q would be the charge on the opposing conductor) and V is the voltage between the conductors. The voltage between conductors from the electric field becomes:

V=−∫E·dl Equation 26.

For an insulated, metallic electrode, the limits of integration are the radius of the inner conductor (d) and the radius of the outer conductor (D). The electric field is in the same direction as the length element (the radial direction), so:

E·dl=|E|dl Equation 27.

The electric field between the inner and outer conductors may be found by Gauss's law:

$$∮ E \cdot da = \frac{A}{∈} . \qquad Equation 28$$

Because the electric field is dependent only on the charge enclosed by a Gaussian surface surrounding the inner conductor, E is not a function of the surface area. Additionally, the field from the conductor is directed radially away from the axis so only the curved surface of the cylinder contributes to the surface integral, the cylinder ends are normal to the field direction so they do not contribute to the dot product. Evaluating the integral:

$$2 π LE = \frac{A}{ϵ} . \qquad Equation 29$$

Solving for the electric field:

$$E = \frac{Q}{2 π {rL}_{ϵ}} . \qquad Equation 30$$

Substituting Equation 30 into Equation 26 and reversing the bounds to remove the negative sign:

$$V = \frac{Q}{2 π L {ϵ}_{0} {ϵ}_{r}} {\int }_{D}^{d} \frac{1}{r} dr = \frac{Q}{2 π L {ϵ}_{0} {ϵ}_{r}} [ ln ( d ) - ln ( D ) ] = \frac{Q}{2 π L {ϵ}_{0} {ϵ}_{r}} ln ( \frac{d}{D} ) , \qquad Equation 31$$

where Q/L is the charge per unit length and ∈=∈₀∈ᵣ, where ∈₀ is the permittivity of free space and ∈ᵣ is the relative permittivity of the insulating medium.

The capacitance as a function of depth (L) in the medium is:

$$C ( L ) = \frac{Q}{V} = \frac{2 π {ϵ}_{0} {ϵ}_{r} L}{ln ( \frac{d}{D} )} . \qquad Equation 32$$

Converting the natural logarithm to base 10 and entering the numerical values for all known constants yields:

$${C}_{S} = \frac{0.245 {ϵ}_{r}}{{log}_{10} ( \frac{d}{D} )} pF \text{/} cm . \qquad Equation 33$$

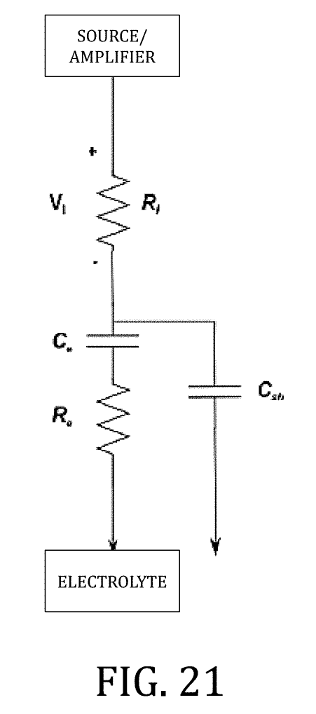

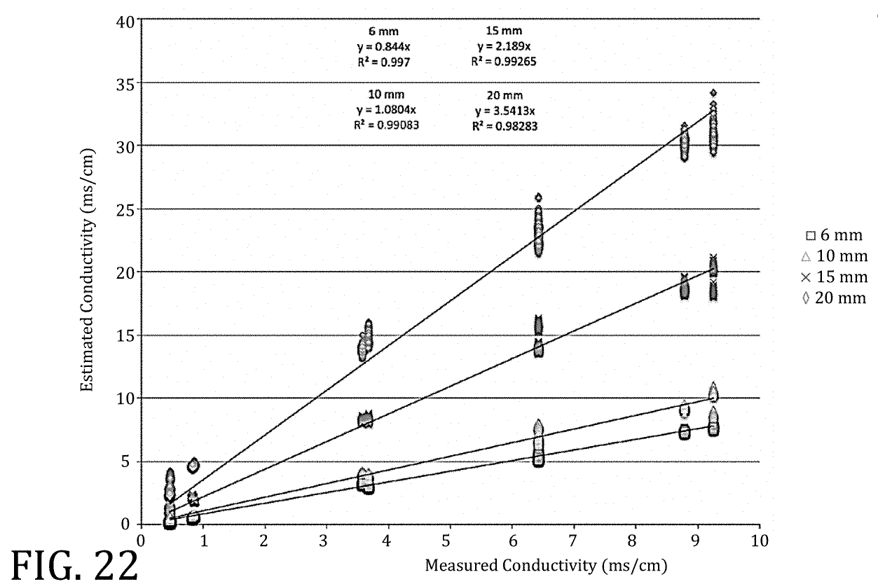

Equation 33 yields a linear increase in shunt capacitance with depth. The resulting electrode circuit, illustrated in FIG. 21, shows that the current (I) measured through the current measurement resistor R₁ is divided between the electrode and the shunt capacitance. As the capacitance increases, the effective resistance (reactance) of the shunt capacitor decreases, reducing the current through the electrode. Calibration of the 4EM system with the electrodes at increasing depth (FIG. 22) shows the slope of the calibration curve increases with depth. The slope changes because the estimated potential is determined from Equation 23 using the current, I, through the resistor, R₁.

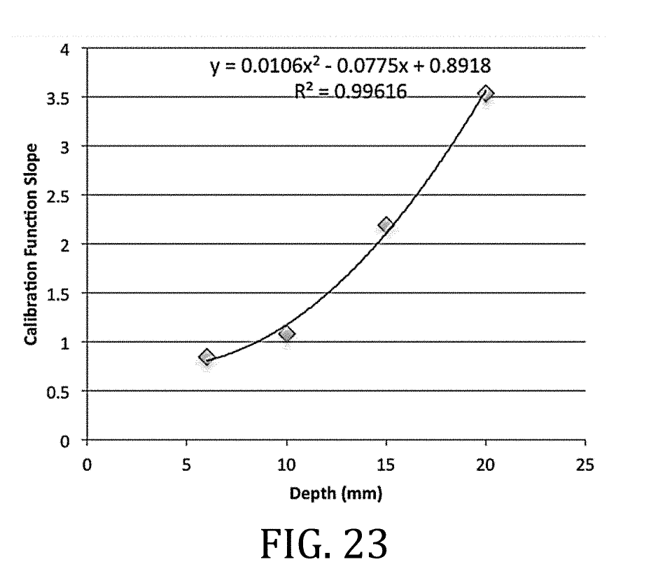

The depth correction is complicated because it depends not only on shunt capacitance, but on interaction of the electric fields in the medium with the air/electrolyte interface and imperfections in the insulating layer on the electrodes. However, an empirical correction curve can be developed by estimating the conductivity of a known solution at a variety of depths in a calibration solution. Depth correction can be applied to prospective measurements by inverting the estimated conductivity equations in FIG. 22 using the slopes from FIG. 23:

$${σ}_{d} = \frac{{σ}_{est}}{{CF}_{d}} , \qquad Equation 34$$

where σ_{d} is the depth corrected estimated conductivity, σₑₛₜ is the raw, estimated conductivity, and CF_{d} is the depth correction function estimated from the slopes of the linear fitted equations in FIG. 22.

For the slopes of the calibration curves in FIG. 22, the depth calibration function (CF_{d}) in FIG. 21 is estimated as:

CF_{d}=0.0106d²−0.0775d+0.8918 Equation 35,

where d is the depth in mm. The calibration function was used for depth correction for measurements by the 4EM.

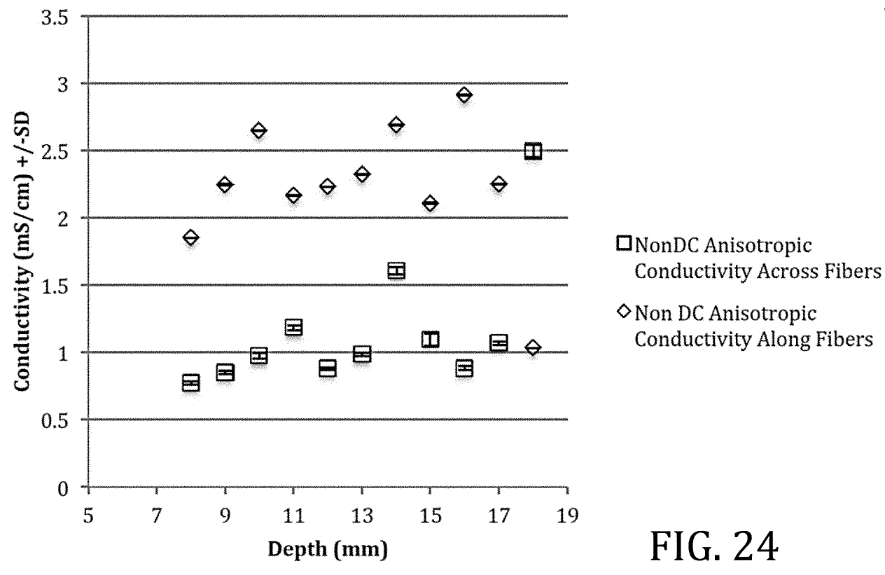

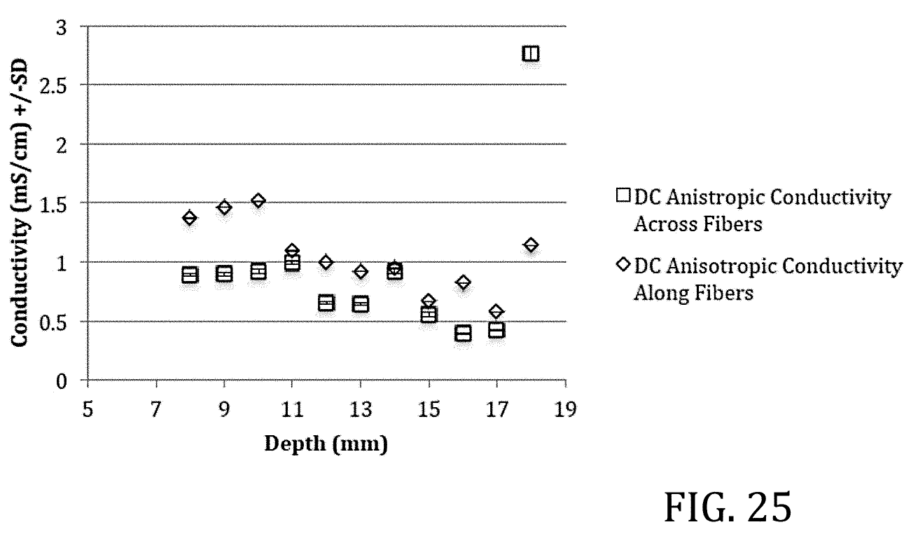

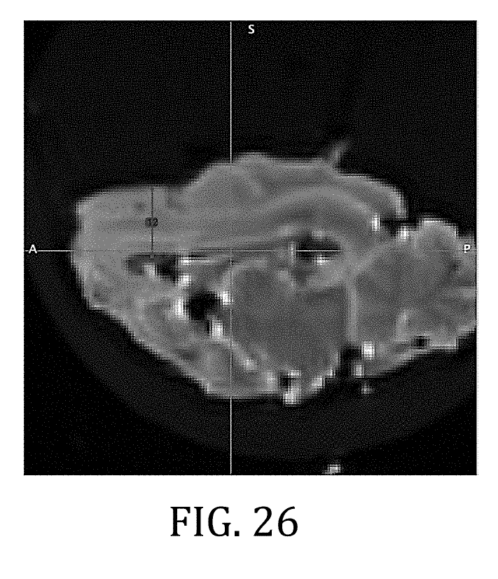

FIG. 24 graphs non depth-corrected conductivity measurement data (derived with Equations 23-25). FIG. 25 graphs the depth-corrected data. The isotropic data at depths of 11 mm, 14 mm, 15 mm, and 17 mm, where the along and across measurement are approximately equal. This agrees with the imaging data (FIG. 26) that shows the center of the corpus callosum at a depth of approximately 12 mm. An inherent complication of measuring conductivity of solid biological tissues arises from the heterogeneous and anisotropic nature of the tissue conductivity.

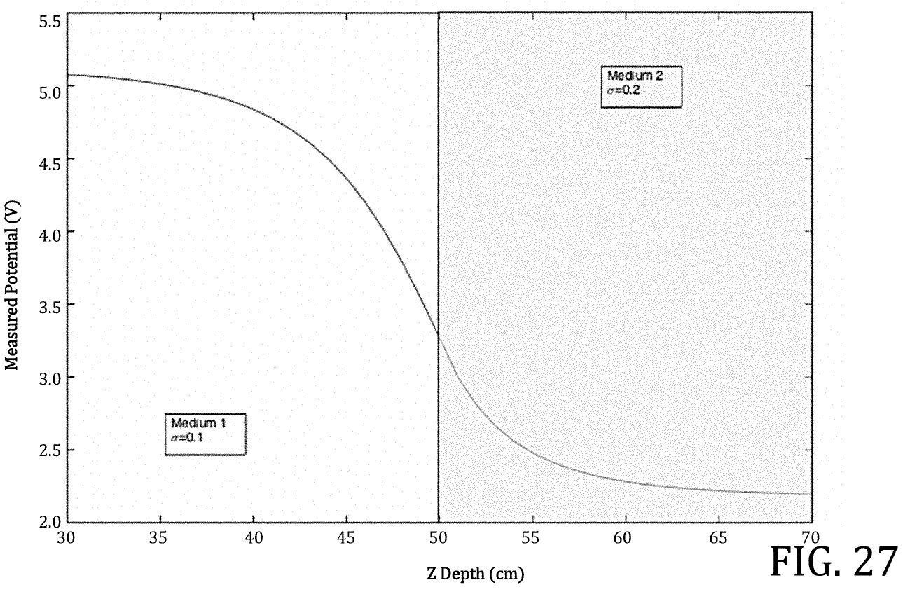

FIG. 27 shows the impact of heterogeneity caused by an interface between two regions of conductive media with differing uniform conductivities. The measured voltage, which ideally would change abruptly at the interface, changes gradually over a range of +/−2 mm from the interface.

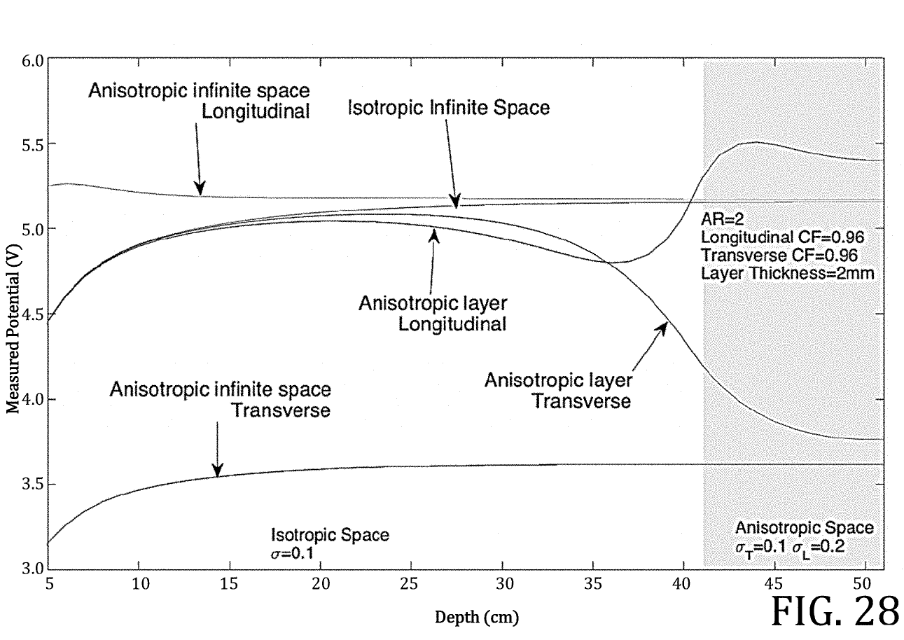

Using the FD model, the effect of a thin anisotropic layer inside an infinite homogenous region may be shown. FIG. 28 demonstrates the effect of an air boundary located at Z=0 and a 2 mm thick anisotropic layer centered at Z=50. The geometry more nearly represents the highly anisotropic conductivity in the corpus callosum and the much less anisotropic conductivity of surrounding tissue. Infinite space conductivity (without a tissue interface) was constant with depth except near the tissue air interface at z=0. The result agrees with Equations 23-25 for isotropic and anisotropic tissue conductivity of an infinite space.

The transverse component of conductivity changed, as shown in FIG. 27 going from a region of high-to-low conductivity. However, the longitudinal component had a dip to the left of the interface and rise to the right, but approached the same value away from the interface. This could lead to drastic changes in the ratio of longitudinal to transverse conductivity around the interface. In fact, the transverse component was slightly larger than the longitudinal component in the region of z=1.5 mm to 3.5 mm. The important characteristics of the model are the anisotropy ratio (“AR”) defined as the ratio between the highest and lowest conductivities of an anisotropic medium, the contrast ratio (“CR”) defined as the ratio between the lowest conductivity of the anisotropic medium and the conductivity of the surrounding isotropic medium, and the depth (thickness) of the anisotropic layer. Longitudinal and transverse geometric correction factors (CF_{gL} and CF_{gT}) for measured potential as a function of layer thickness, AR, and CR allow a more precise estimate of conductivity within the corpus callosum tissue.

A fit of the FD model to a measured data set provides a basis for estimating CF_{gL}, and CF_{gT}.

The following equations for anisotropic conductivity at the center of an anisotropic layer with depth (CF_{d}) and geometric (CF_{gL} and CF_{gT}) corrections:

$$\begin{matrix}{σ}_{T} = \frac{1}{4 π a {ϕ}_{L} {CF}_{gT} {CF}_{d}} , and & Equation 36 \\ {σ}_{L} = \frac{1}{4 π a {ϕ}_{T}^{2} {CF}_{gT} {CF}_{d}} , & Equation 37\end{matrix}$$

where φ_{T} and φ_{L} are the measured potentials transverse and longitudinal to the anisotropic structure of the tissue.

FIG. 26 is a T2 MRI sagittal image of a pig brain with voxel size of 1 mm×1 mm×1 mm. Layer thickness of the corpus callosum (the dark horizontal layer) was estimated at the conductivity measurement location. The corpus callosum is a discrete structure within the brain that should appear with sharp edges; however, the voxels at the edge of the corpus callosum contain both corpus callosum and non-corpus callosum tissue due to a partial volume effect. Therefore, the thickness of the corpus callosum was estimated to be about 2 mm. The thickness of the corpus callosum in this image was estimated to be 2.5 mm, which would be verified by an examination of the anisotropic region from the measured data.

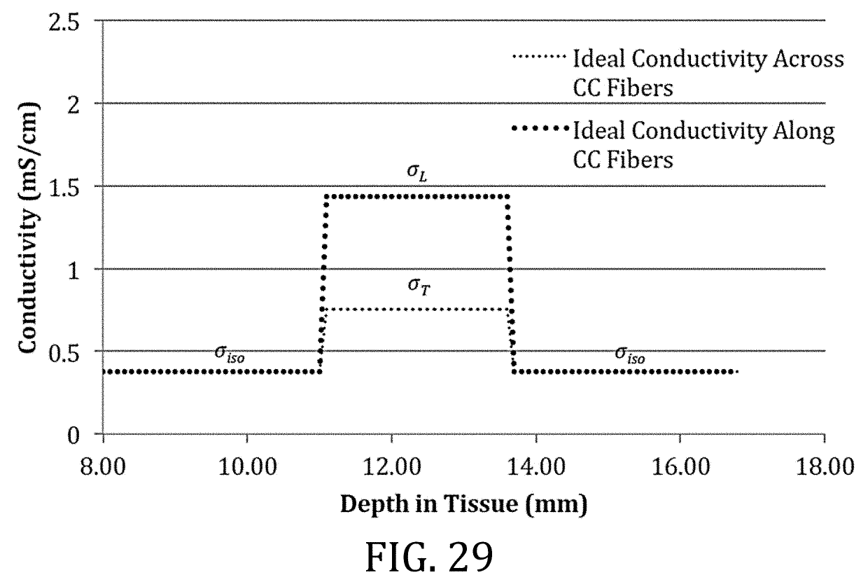

The region of the brain local to the corpus callosum was modeled as an idealized anisotropic layer characterized by a conductivity (ϕ_{L}) along the direction of greatest conductivity and a conductivity (ϕ_{T}) along the direction of least conductivity (FIG. 29). The anisotropic layer was flanked by regions of isotropic conductivity (ϕᵢₛₒ). The important parameters of the model are anisotropy ratio (ϕ_{L}/ϕ_{T}), the contrast ratio (ϕᵢₛₒ/ϕ_{T}), and the thickness of the anisotropic layer.

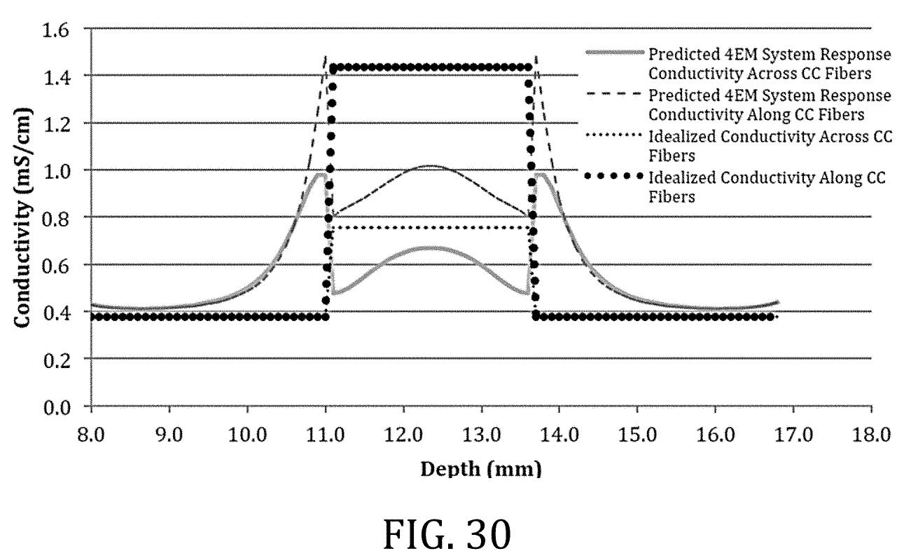

FIG. 30 shows the idealized conductivities and the 4EM system response to the conductivities as the electrodes are moved through the anisotropic layer.

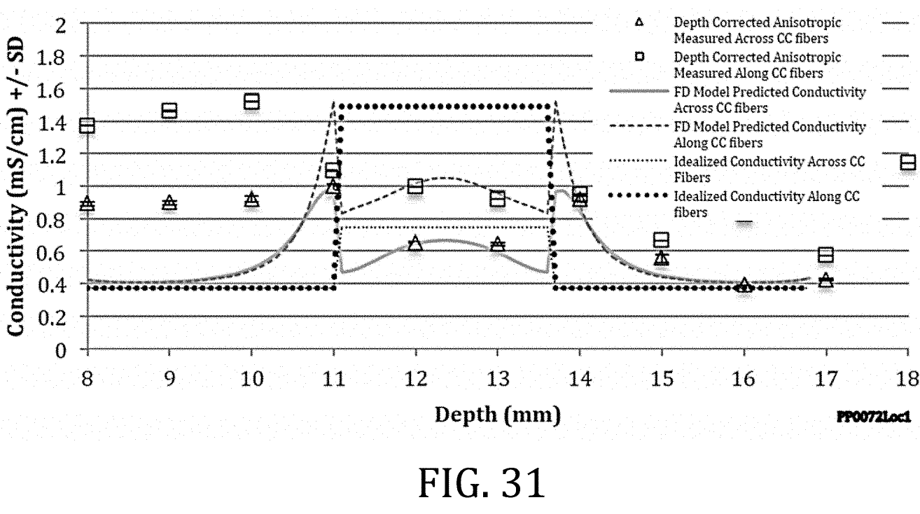

FIG. 31 shows a measured dataset overlaid with an FD model prediction of the 4EM system response based on measurement of a tissue geometric model with idealized longitudinal and transverse conductivities. CF_{gL} and CF_{gT} are the ratios of the predicted conductivity to the idealized conductivity at the measurement location in the longitudinal and transverse directions. The measured conductivities at depths of 8 mm to 10 mm did not match the model due to the presence of the longitudinal fissure, which was not included in this idealized model.

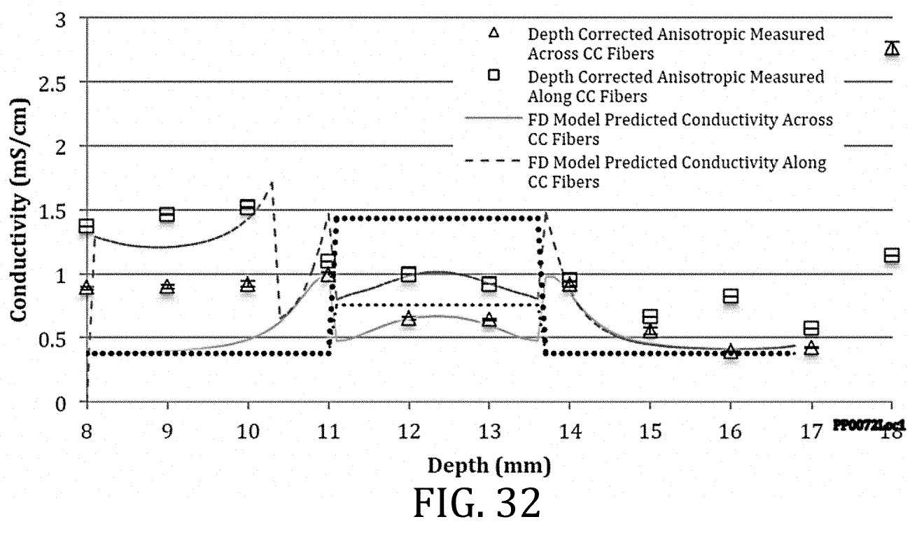

FIG. 32 is similar to FIG. 29 but further incorporates a rudimentary, non-optimized longitudinal fissure model into the geometric model. The artifact induced elevation of the measured conductivities along the corpus callosum fibers, across the longitudinal fissure, follows the general trend of the model.

The variability of the 4EM system measurement was derived from the variability of 20 repeated measurements assuming the noise is normally distributed. The number of measurements (N=20) at each data point was not based on a rigorous power analysis, but rather was coded into the MATLAB code early in the development of the 4EM system and before a stable configuration was found. Once the system was finalized, N was left at a value of 20 because it was not a burden on computer system resources. The error bars on the final measurements are very small, and a power analysis would likely show that N could be reduced.

The small variability in the measurements stems from electronic noise in the system as the measurement period was short enough to prevent drift from being a factor in the measurements. Repeated pre- and post-calibrations showed instrument drift was not an important factor in the results of the measurements.

### Example 5—Comparison of Model and Measurement

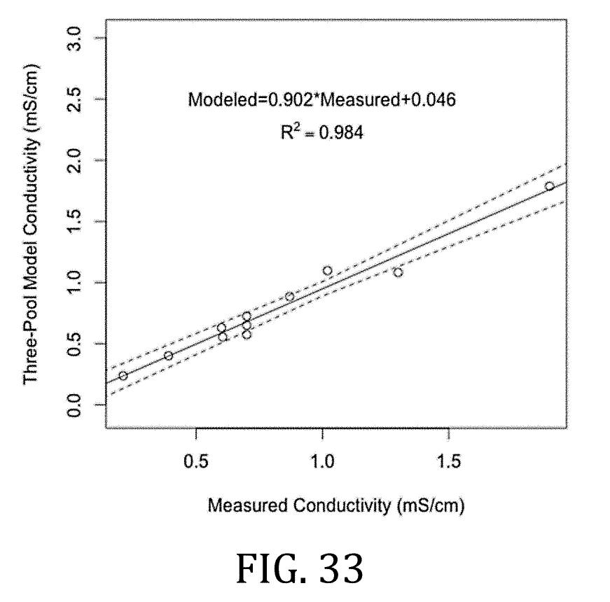

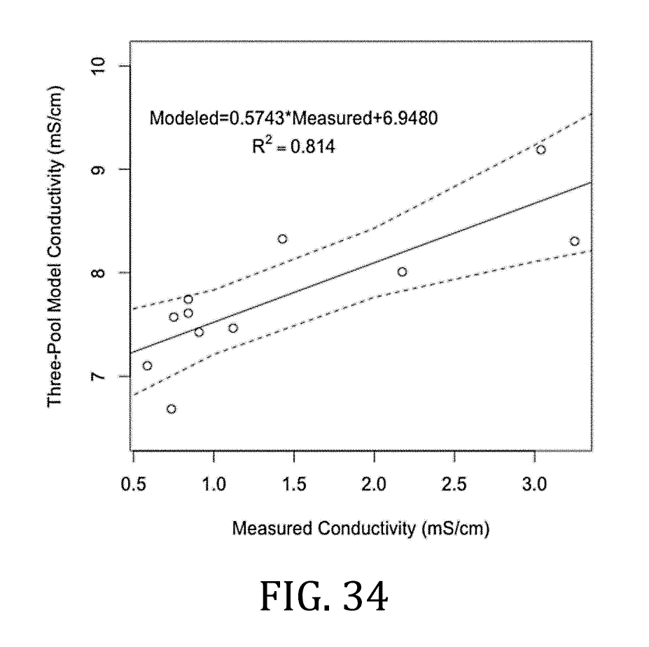

The results of the three-pool model conductivity prediction and the measured conductivity at the center of the corpus callosum may be compared to determine if the three-pool model, according to the various embodiments described herein, may be used to map conductivity in a clinical or research application. FIGS. 33 and 34 graphically illustrate conductivity in a first direction, across an axon, and a second direction, along an axon, as calculated by the three-pool model versus measured conductivity from the 4EM system. The confidence bounds on the plot are a 95% confidence interval on the fit of the central line.

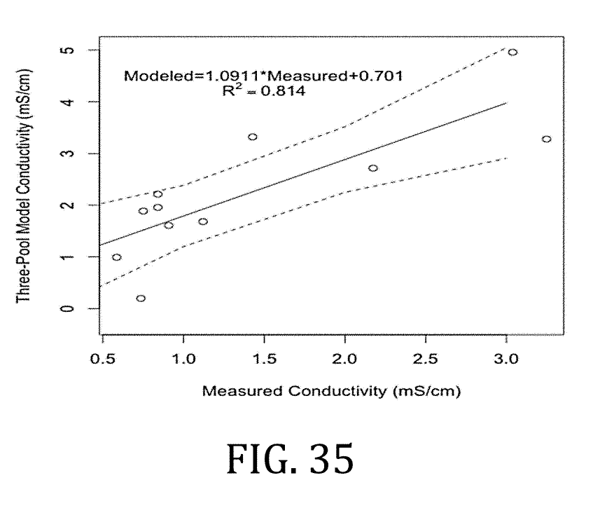

If embodiments of the three-pool model approximate direct measurement, then FIGS. 33 and 34 would demonstrate lines having slopes approaching unity. Indeed, FIG. 33 (across the axons measurement) has a slope close to unity and a high correlation (R²=0.98). FIG. 34 (along the axons measurement) has a linear characteristic; however, the slope is not close to unity, which may be due to a lack of glial cells in the model. A simplistic linear transformation (in essence, a calibration factor), f_{mxT}=1.74(fₘₓ−0.41) corrects the slope of the curve with a reasonably high correlation (R²=0.814) as shown in FIG. 35.

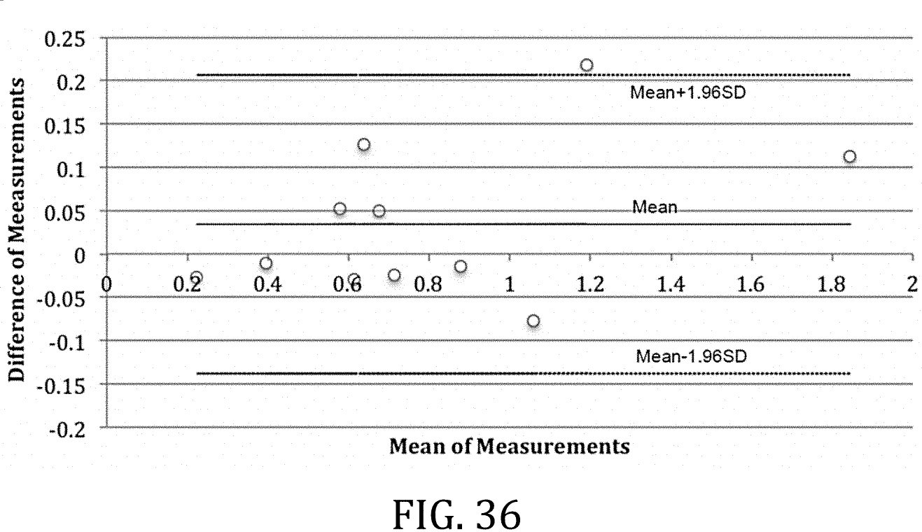

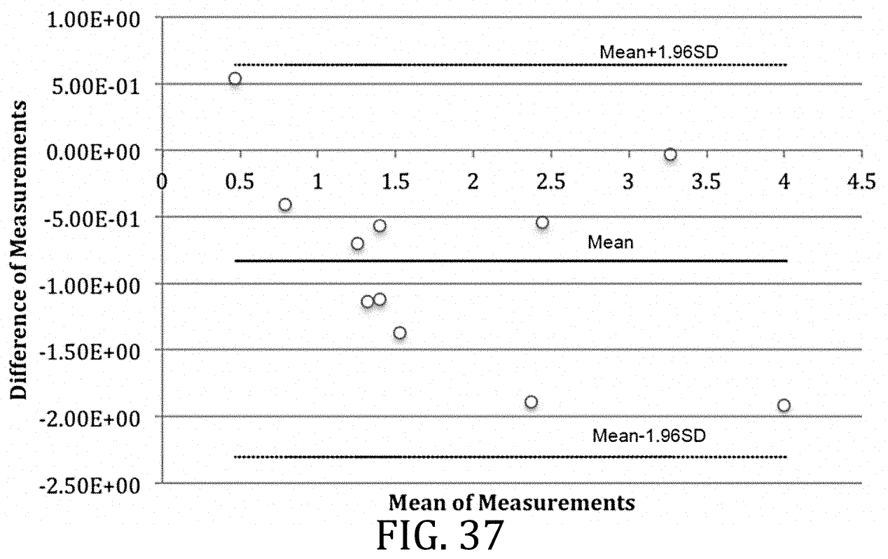

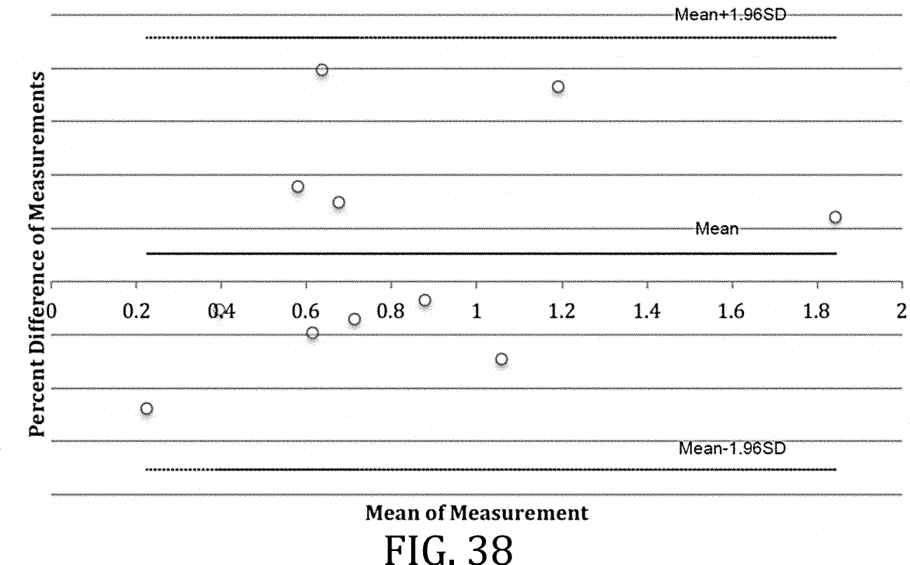

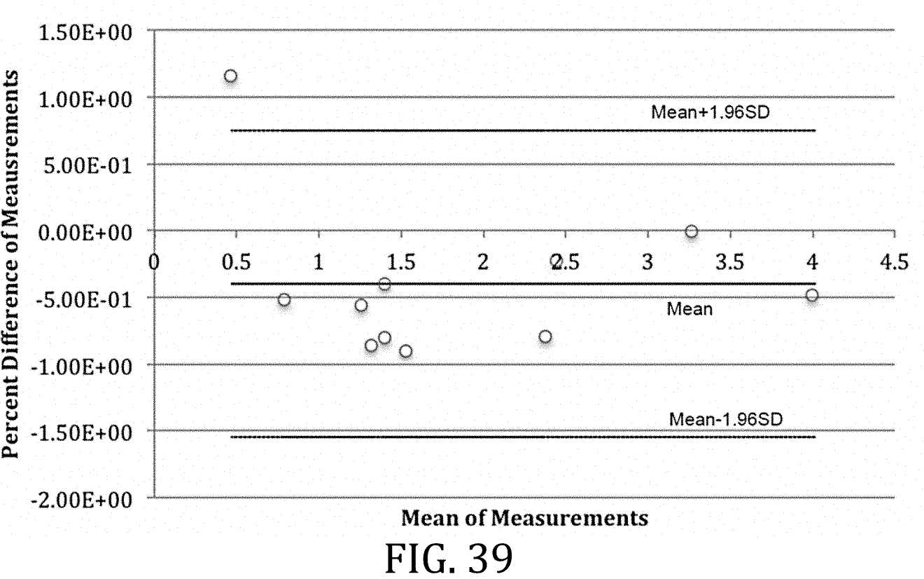

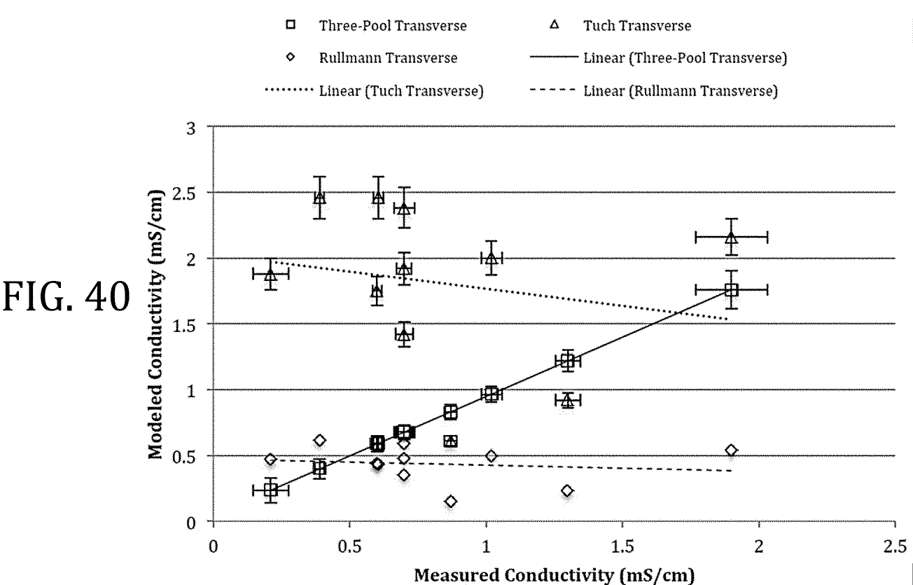

FIG. 36 is a graphical representation of a Bland-Altman analysis in an across direction (J. M. BLAND et al., “Characterization and propagation of uncertainty in diffusion-weighted MR imaging,” MRM, Vol. 50 (2003) 1077-1088); FIG. 37 is the Bland-Altman analysis in an along direction. The variability of the measurements increases with increasing mean measurement. Some have shown that Bland-Altman using percent differences removes the effect of increasing variability with measured amplitude; therefore, FIGS. 38 and 39 show the Bland-Altman analysis with percent differences. With the exception of one outlier in FIG. 40, all measurements are within the 95% confidence bounds.

Accepting the Bland-Altman analysis as evidence that the three-pool model is a fairly good predictor of white matter conductivity, the three pool model was compared with two conventional methods: (1) D. S. TUCH et al., “Error analysis of tissue resistivity measurement,” IEEE Transactions on Bio-Medical Engineering, Vol. 49 (2001) 41-48, and (2) M. RULLMAN et al., “EEG source analysis of epileptiform activity using a 1 mm anisotropic hexahedra finite element head model,” Neuroimaging, Vol. 44 (2009) 399-410. The three-pool model assumes rotational symmetry so that there is a single value for the transverse direction (ϕ_{T}). The conventional methods produce one value for each eigenvalue, so the shorter of the two eigenvalues would be averaged to yield a representative transverse value.

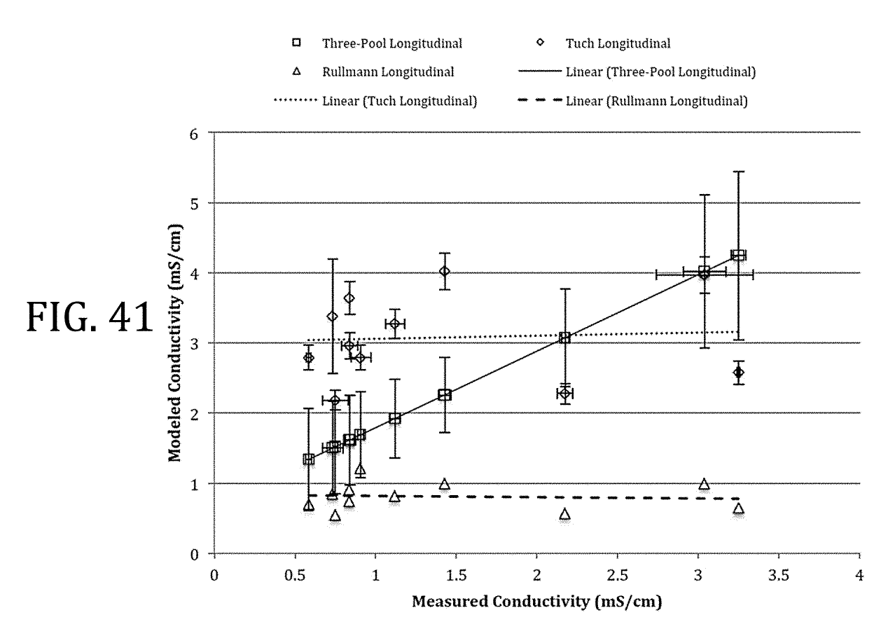

Table 20, below, is a comparison of the three methods. FIGS. 40 and 41 graphically compare the three methods, with neither the Rullmann or Tuch methods yielding results similar to the Three-Pool model.

The three-pool model estimation of extra axonal space can be used to predict conductivity in the central corpus callosum. Conductivity across the fiber bundle was predicted without calibration, and the Bland-Altman analysis showed the results of the prediction to be equivalent to direct measurement. Along the fiber direction, the prediction was linear, but required a correction factor for the presence of glial cells to provide an equivalent value.

The Tuch and Rullmann methods did not produce results that compared well with the three-pool model results or the other measured values. The differences were attributed to partial volume effect in the diffusion images or to the limitations of the Gaussian assumption of particle diffusion underlying the diffusion imaging technique. Regardless, the three-pool model method was found to be applicable to practical clinical or research settings where a conductivity map of the brain is desired.

TABLE 20

| | LOC/ | Three-Pool | Tuch | |
|---|---|---|---|---|
| Subject | ROI | (±2SD) | (±2SD) | Rullmann |
| PP0016 | 1/9 | σ_{T} = 0.83 ± 0.055 | σ_{T} = 0.61 ± 0.039 | σ_{T} = 0.153 |
| | | σ_{L} = 3.07 ± 0.694 | σ_{L} = 2.28 ± 0.147 | σ_{L} = 0.567 |
| PP0016 | 2/26 | σ_{T} = 1.75 ± 0.144 | σ_{T} = 2.16 ± 0.140 | σ_{T} = 0.538 |
| | | σ_{L} = 4.01 ± 1.090 | σ_{L} = 3.97 ± 0.26 | σ_{L} = 0.989 |
| PP0017 | 1/9 | σ_{T} = 0.59 ± 0.060 | σ_{T} = 1.75 ± 0.11 | σ_{T} = 0.436 |
| | | σ_{L} = 1.52 ± 0.667 | σ_{L} = 2.18 ± 0.141 | σ_{L} = 0.544 |
| PP0017 | 2/12 | σ_{T} = 0.40 ± 0.076 | σ_{T} = 2.46 ± 0.16 | σ_{T} = 0.613 |
| | | σ_{L} = 1.33 ± 0.729 | σ_{L} = 2.79 ± 0.18 | σ_{L} = 0.693 |
| PP0063 | 1/12 | σ_{T} = 0.59 ± 0.060 | σ_{T} = 2.46 ± 0.16 | σ_{T} = 0.432 |
| | | σ_{L} = 1.69 ± 0.617 | σ_{L} = 2.79 ± 0.18 | σ_{L} = 1.21 |
| PP0063 | 2/13 | σ_{T} = 0.677 ± 0.056 | σ_{T} = 2.38 ± 0.154 | σ_{T} = 0.592 |
| | | σ_{L} = 1.62 ± 0.637 | σ_{L} = 2.96 ± 0.191 | σ_{L} = 0.737 |
| PP0065 | 1/14 | σ_{T} = 1.21 ±0.080 | σ_{T} = 0.924 ± 0.059 | σ_{T} = 0.229 |
| | | σ_{L} = 4.25 ± 1.20 | σ_{L} = 2.58 ± 0.167 | σ_{L} = 0.643 |
| PP0065 | 2/10 | σ_{T} = 0.677 ± 0.056 | σ_{T} = 1.42 ± 0.092 | σ_{T} = 0.353 |
| | | σ_{L} = 1.92 ± 0.566 | σ_{L} = 3.27 ± 0.211 | σ_{L} = 0.814 |
| PP0066 | 1/9 | σ_{T} = 0.235 ± 0.092 | σ_{T} = 1.88 ± 0.121 | σ_{T} = 0.468 |
| | | σ_{L} = 1.50 ± 0.672 | σ_{L} = 3.38 ± 0.818 | σ_{L} = 0.840 |
| PP0066 | 2/13 | σ_{T} = 0.677 ± 0.056 | σ_{T} = 1.92 ± 0.124 | σ_{T} = 0.478 |
| | | σ_{L} = 1.62 ± 0.637 | σ_{L} = 3.64 ± 0.234 | σ_{L} = 0.905 |
| PP0072 | 2/14 | σ_{T} = 0.966 ± 0.060 | σ_{T} = 2.00 ± 0.129 | σ_{T} = 0.497 |
| | | σ_{L} = 2.26 ± 0.537 | σ_{L} = 4.02 ± 0.260 | σ_{L} = 0.990 |

where Loc/ROI refers to the location in the brain and the ROI number in the array of ROIs about the presumed measurement location.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

What is claimed is:

1. A method of mapping tissue conductivity, the method comprising:
estimating an extra-axonal fraction using T1- and T2-weighted MRI; and
assigning longitudinal and transverse flow directions using diffusion-weighted MRI.

2. The method of claim 1, wherein estimating the extra-axonal fraction further comprises:
estimating an axonal fraction; and
estimating a myelin fraction.

3. The method of claim 2, wherein the extra-axonal, axonal, and myelin fractions are calculated according to:
$$[ \begin{matrix}{f}_{my} \\ {f}_{ma} \\ {f}_{mx}\end{matrix} ] = {[ \begin{matrix}1 & 1 & 1 \\ w {1}_{my} & w {1}_{ma} & w {1}_{mx} \\ w {2}_{my} & w {2}_{ma} & w {2}_{mx}\end{matrix} ]}^{- 1} [ \begin{matrix}1 \\ 1 \\ 0\end{matrix} ]$$
wherein the axonal fraction is fₘₐ, the myelin fraction is f_{my}, the extra-axonal fraction is fₘₓ, the w1 weighting factors are calculated from measured longitudinal relaxation rates, and the w2 weighting factors are calculated from transverse relaxation rates.

4. The method of claim 3, wherein estimating the axonal, myelin, and extra-axonal fractions further comprises:
$${f}_{my} w {1}_{my} + {f}_{ma} w {1}_{ma} + {f}_{mx} w {1}_{mx} = 1 ;$$
$${S}_{PDW} = S ( 0 ) [ {f}_{my} {e}^{- ( \frac{TE 1}{T {2}_{my}} )} + {f}_{ma} {e}^{- ( \frac{TE 1}{T {2}_{ma}} )} + {f}_{mx} {e}^{- ( \frac{TE 1}{T {2}_{mx}} )} ] ;$$
$${S}_{T 2 W} = S ( 0 ) [ {f}_{my} {e}^{- ( \frac{TE 2}{T {2}_{my}} )} + {f}_{ma} {e}^{- ( \frac{TE 2}{T {2}_{ma}} )} + {f}_{mx} {e}^{- ( \frac{TE 2}{T {2}_{mx}} )} ] ; and$$
$${f}_{my} w {2}_{my} + {f}_{ma} w {2}_{ma} + {f}_{mx} w {2}_{mx} = 0.$$

5. The method of claim 1, wherein assigning longitudinal and transverse flow directions further comprises:
deriving eigenvectors and eigenvalues from the diffusion-weighted magnetic resonance imaging using a three-dimensional Gaussian fit.

6. The method of claim 5, wherein the eigenvectors and eigenvalues define a direction ellipse.

7. The method of claim 1, further comprising:
reducing or removing at least one image artifact from the plurality of T1-weighted images, the plurality of T2-weighted images, the plurality of diffusion-weighted images, or a combination thereof.

8. A method of mapping biological conductivity, the method comprising:
acquiring a plurality of T1-weighted images a biological tissue;
acquiring a plurality of T2-weighted images of the biological tissue;
acquiring a plurality of diffusion-weighted images of the biological tissue;
estimating an extra-axonal fraction of a region of interest using the pluralities of T1- and T2-weighted images; and
estimating a longitudinal direction of anisotropy and a transverse direction of anisotropy for the region of interest using the plurality of diffusion-weighted images.

9. The method of claim 8, wherein the region of interest is an image pixel.

10. The method of claim 8, wherein the region of interest is a plurality of adjacent pixels.

11. The method of claim 8, wherein estimating the extra-axonal fraction further comprises:
estimating an axonal fraction; and
estimating a myelin fraction.

12. The method of claim 11, wherein the extra-axonal, axonal, and myelin fractions are calculated according to:
$$[ \begin{matrix}{f}_{my} \\ {f}_{ma} \\ {f}_{mx}\end{matrix} ] = {[ \begin{matrix}1 & 1 & 1 \\ w {1}_{my} & w {1}_{ma} & w {1}_{mx} \\ w {2}_{my} & w {2}_{ma} & w {2}_{mx}\end{matrix} ]}^{- 1} [ \begin{matrix}1 \\ 1 \\ 0\end{matrix} ]$$
wherein the axonal fraction is fₘₐ, the myelin fraction is f_{my}, the extra-axonal fraction is fₘₓ, the w1 weighting factors are calculated from the plurality of T1-weighted images, and the w2 weighting factors are calculated from the plurality of T2-weighted images.

13. The method of claim 12, wherein estimating the first, second, and third fractions further comprises:
$${f}_{my} w {1}_{my} + {f}_{ma} w {1}_{ma} + {f}_{mx} w {1}_{mx} = 1 ;$$
$${S}_{PDW} = S ( 0 ) [ {f}_{my} {e}^{- ( \frac{TE 1}{T {2}_{my}} )} + {f}_{ma} {e}^{- ( \frac{TE 1}{T {2}_{ma}} )} + {f}_{mx} {e}^{- ( \frac{TE 1}{T {2}_{mx}} )} ] ;$$
$${S}_{T 2 W} = S ( 0 ) [ {f}_{my} {e}^{- ( \frac{TE 2}{T {2}_{my}} )} + {f}_{ma} {e}^{- ( \frac{TE 2}{T {2}_{ma}} )} + {f}_{mx} {e}^{- ( \frac{TE 2}{T {2}_{mx}} )} ] ; and$$
$${f}_{my} w {2}_{my} + {f}_{ma} w {2}_{ma} + {f}_{mx} w {2}_{mx} = 0.$$

14. The method of claim 8, wherein estimating the longitudinal and transverse directions of anisotropy comprises:
deriving eigenvectors and eigenvalues from the plurality of echo planar images using a three-dimensional Gaussian fit.

15. The method of claim 14, wherein the eigenvectors and eigenvalues define a direction ellipse.

16. The method of claim 8, further comprising:
reducing or removing at least one image artifact from the plurality of T1-weighted images, the plurality of T2-weighted images, the plurality of diffusion-weighted images, or a combination thereof.
